(19) United States
(12) Patent Application Publication
(10) Pub. No.: US 2023/0008098 A1
(43) Pub. Date: Jan. 12, 2023

(54) VISUALIZATION OF THE ANTIGEN BINDING PROFILE FOR A SET OF CLONOTYPES

(71) Applicant: 10x Genomics, Inc., Pleasanton, CA (US)

(72) Inventor: Michael John Terry Stubbington, Cambridge (GB)

(21) Appl. No.: 17/752,679

(22) Filed: May 24, 2022

Related U.S. Application Data
(60) Provisional application No. 63/192,558, filed on May 24, 2021.

(51) Int. Cl. G16B 45/00; C12Q 1/6881

(52) U.S. Cl. CPC G16B 45/00; C12Q 1/6881; C12Q 2600/156

(57) ABSTRACT

A method for visualizing multi-antigen binding capabilities of a set of clonotype groups. Clonotype data that identifies a clonotype group derived from an immune cell sequence dataset (e.g., single cell or spatial dataset) is obtained. A set of interactions for the clonotype group is identified. An interaction in the set of interactions is between a set of cells in the clonotype group and a plurality of antigens in which each cell of the set of cells binds to the plurality of antigens. A binding diagram is generated for the clonotype group based on the set of interactions that has been identified. The binding diagram includes a set of interaction representations that visually represents the set of interactions for the clonotype group. An interaction representation in the set of interaction representations visually relates the plurality of antigens and visually indicates a number of cells in the set of cells that bind to the plurality of antigens.

### CROSS-REFERENCE TO RELATED APPLICATIONS

This application claims priority to and the benefit of U.S. Provisional Application No. 63/192,558, filed May 24, 2021, the contents of this is incorporated herein in its entirety.

### BACKGROUND

The immune system recognizes and eliminates non-self threats through a complex and layered network of both innate and adaptive immune cells. Robust characterization of this response and discovery of novel cell types and antigen-specific populations has proven challenging to perform in a high-throughput fashion due to the single-modal nature of flow cytometry, CyTOF, and similar assays. One example of an approach to addressing the shortcomings of flow cytometry is to utilize multi-modal droplet-based single cell technologies to analyze pre- and post-vaccination T cells, B cells, and peripheral blood mononuclear cells from influenza vaccines or other vaccines (or of samples collected over time from individuals affected by diseases such as systemic lupus erythematosus and other autoimmune disorders, chronic viral infection, and acute/non-chronic viral infection).

Using this framework, T cell and B cell responses (e.g., vaccine-specific T cell and/or B cell responses) can be identified and used to implement an immune cell (e.g., B cell, T cell, peripheral blood mononuclear cell (PBMC), etc.) clonotyping algorithm. The clonotyping algorithm may, for example, resolve post-vaccination, post-disease, or post-treatment activated immune cell antibody lineages at scale by combining untargeted and targeted gene expression, full-length immune cell receptor sequencing, surface protein expression and/or antigen capture, in addition to tag-based and genetic demultiplexing.

Further, it may be desirable to understand the antigen binding properties of the immune cells in a particular clonotype (or clonotype group) or a set of clonotypes. Identifying cells that bind to multiple antigens and understanding the multi-antigen binding of the various cells in a clonotype may aid in the domains of antibody discovery, characterization, and antibody engineering, where assignment of the correct clonotypes is foundational to understanding how alterations in cell phenotype and antigen specificity are linked in immunotherapeutic products, passive and active vaccinations, and ease of engineering (presence or absence of glycosylation sites, addition or reversion of mutations in antibody lineages, etc.).

### SUMMARY

In one or more embodiments, a method for visualizing immune cells within an immune cell receptor dataset is provided. The immune cell receptor dataset is obtained from a sample. The immune cell receptor dataset includes a plurality of immune cell receptor sequences. Each immune cell receptor sequence is associated with an individual immune cell in the sample. A clonotype group comprising a set of individual immune cells is obtained from the immune cell receptor dataset. At least one interaction between at least one cell of the set of individual immune cells in the clonotype group and a plurality of antigens is identified. A visualization schema is selected to visualize the at least one interaction. A visualization of the clonotype group is rendered according to the visualization schema. The visualization displays the at least one interaction.

In one or more embodiments, a computer-program product is tangibly embodied in a non-transitory machine-readable storage medium, including instructions configured to cause one or more data processors to perform a method for visualizing immune cells within an immune cell receptor dataset. The method comprises obtaining the immune cell receptor dataset from a sample, the immune cell receptor dataset including a plurality of immune cell receptor sequences, wherein each immune cell receptor sequence is associated with an individual immune cell in the sample; obtaining a clonotype group comprising a set of individual immune cells from the immune cell receptor dataset; identifying at least one interaction between at least one cell of the set of individual immune cells in the clonotype group and a plurality of antigens; selecting a visualization schema to visualize the at least one interaction; and rendering a visualization of the clonotype group according to the visualization schema, wherein the visualization displays the at least one interaction.

In one or more embodiments, a visualization system is provided. The visualization system includes a processing unit, a display unit, and a data source configured to obtain an immune cell receptor dataset from a sample, the immune cell receptor dataset including a plurality of immune cell receptor sequences. Each immune cell receptor sequence is associated with an individual immune cell in the sample. The processing unit is communicatively connected to the data source and configured to receive the immune cell receptor dataset. The processing unit comprises a clonotype grouping engine configured to obtain a clonotype group comprising a set of individual immune cells from the immune cell receptor dataset; an interaction identification engine configured to identify at least one interaction between at least one cell of the set of individual immune cells in the clonotype group and a plurality of antigens; and a schema selection engine configured to select a visualization schema to visualize the at least one interaction. The display unit is configured to render a visualization of the clonotype group according to the visualization schema, wherein the visualization displays the at least one interaction.

In various embodiments, a method is provided for visualizing multi-antigen binding capabilities of a set of clonotype groups. Clonotype data that identifies a clonotype group derived from an immune cell sequence dataset is obtained. A set of interactions for the clonotype group is identified. An interaction in the set of interactions is between a set of cells in the clonotype group and a plurality of antigens in which each cell of the set of cells binds to the plurality of antigens. A binding diagram is generated for the clonotype group based on the set of interactions that has been identified. The binding diagram includes a set of interaction representations that visually represents the set of interactions for the clonotype group. An interaction representation in the set of interaction representations visually relates the plurality of antigens and visually indicates a number of cells in the set of cells that bind to the plurality of antigens.

These and other aspects and implementations are discussed in detail herein. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 is a schematic diagram of a workflow for single cell sequencing in accordance with various embodiments.

FIG. 2 is a schematic diagram of a workflow for clonotyping in accordance with one or more embodiments.

FIG. 3 is a schematic diagram of a visualization system in accordance with one or more embodiments.

FIG. 4 is an illustration of a binding diagram in accordance with one or more embodiments.

FIG. 5 is an illustration of a binding diagram from FIG. 4 with additional information in accordance with one or more embodiments.

FIG. 6 is an illustration of a binding diagram from FIG. 5 with additional information in accordance with one or more embodiments.

FIGS. 7A and 7B is an illustration of other binding diagrams in accordance with one or more embodiments.

FIGS. 8A and 8B is an illustration of other binding diagrams in accordance with one or more embodiments.

FIGS. 9A and 9B is an illustration of other binding diagrams in accordance with one or more embodiments.

FIG. 10 is a flowchart of a method for visualizing immune cells within an immune cell receptor dataset in accordance with various embodiments.

FIG. 11 is a flowchart of a method for visually presenting antigen binding profiles of cells in accordance with one or more embodiments.

FIG. 12 is a block diagram that illustrates a computer system in accordance with various embodiments.

FIG. 13 is a schematic diagram showing an exemplary capture probe, in accordance with various embodiments.

FIG. 14 is a schematic illustrating a cleavable capture probe, wherein the cleaved capture probe can enter into a non-permeabilized cell and bind to analytes within the sample, in accordance with various embodiments.

FIG. 15 is a schematic diagram of an exemplary multiplexed spatially-barcoded feature, in accordance with various embodiments.

FIG. 16A is a schematic diagram illustrating an exemplary embodiment of a spatial methodology for generating immune cell data (e.g., sequence data for an antigen binding molecule (ABM), in accordance with various embodiments.

FIG. 16B is a schematic diagram illustrating an exemplary embodiment of a spatial methodology for generating immune cell data, in accordance with various embodiments.

FIG. 17 is a schematic diagram illustrating an exemplary analyte enrichment strategy following analyte capture on the array, in accordance with various embodiments.

FIG. 18 is a schematic diagram illustrating a sequencing strategy with a primer specific complementary to the sequencing flow cell attachment sequence (e.g., P5) and a custom sequencing primer complementary to a portion of the constant region of the analyte, in accordance with various embodiments.

FIG. 19 is a schematic diagram illustrating an exemplary nucleic acid library preparation method to remove a portion of an analyte sequence via double circularization of a member of a nucleic acid library, in accordance with various embodiments.

FIG. 20 is a schematic diagram illustrating another exemplary workflow for processing such double-stranded circularized nucleic acid product, in accordance with various embodiments.

FIG. 21 is a schematic diagram illustrating an exemplary nucleic acid library preparation method to remove all or a portion of a constant sequence of an analyte from a member of a nucleic acid library via circularization, in accordance with various embodiments.

FIG. 22 is a schematic diagram illustrating an exemplary nucleic acid library preparation method to reverse the orientation of an analyte sequence in a member of a nucleic acid library, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### Introduction

The following description of various embodiments is exemplary and explanatory only and is not to be construed as limiting or restrictive in any way. Other embodiments, features, objects, and advantages of the present teachings will be apparent from the description and accompanying drawings, and from the claims.

It should be understood that any use of subheadings herein is for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features is used, largely for informational purposes, and not in any way to limit the design, subfeature, and functionality of the specifically described feature.

Any publication mentioned herein is incorporated by reference herein in its entirety for the purpose of describing and disclosing devices, compositions, formulations, and methodologies which are described in the publication and which might be used in connection with the embodiments described herein.

The various embodiments described herein provide methods and systems for providing a visualization of the antigen binding profiles of immune cells. An antigen binding profile for an immune cell is an identification of the one or more antigens that the immune cell is capable of binding. In one or more embodiments, this visualization is provided with respect to a clonotype or clonotype group. In some embodiments, this visualization is provided for multiple clonotypes. In various embodiments, the visualization is generated in the form of one or more binding diagrams that can be displayed to a user to enable the user to readily and easily discern important information about the multi-antigen binding capabilities of a given clonotype, a group of clonotypes, or both.

In one or more embodiments, the visualization of the antigen binding profiles for the cells in a clonotype group includes a binding diagram that identifies the multi-antigen binding capabilities of the various cells in that clonotype group. The binding diagram uses visual or graphical features (e.g., graphical representations, graphical indicators, graphical components, etc., or combination thereof) to visually represent the multi-antigen binding capabilities of the clonotype group in a manner that is readily and easily discernible by a user. The visualization system described herein by the various embodiments enables generating binding diagrams for multiple clonotypes in an efficient manner that reduces the overall computing resources and visual space that would otherwise be needed to present such information to a user give the sheer number (e.g., hundreds, thousands, etc.) of cells that may be in a particular clonotype group. Further, these binding diagrams reduce the complexity with respect to how this information is presented to a user.

### II. Definitions & Exemplary Context

As used herein, the terms “comprise,” “comprises,” “comprising,” “contain,” “contains,” “containing,” “have,” “having,” “include,” “includes,” and “including” and their variants are not intended to be limiting, are inclusive or open-ended, and do not exclude additional, unrecited additives, components, integers, elements, or method steps. For example, a process, method, system, composition, kit, or apparatus that comprises a list of features is not necessarily limited only to those features but may include other features not expressly listed or inherent to such process, method, system, composition, kit, or apparatus.

Unless otherwise required by context, singular terms shall include pluralities and plural terms shall include the singular.

Unless defined otherwise, all scientific and technical terms used herein with respect to the various embodiments described herein have the same meaning as commonly understood by those of ordinary skill in the art.

Generally, the nomenclatures, techniques, and laboratory procedures described herein (e.g., in connection with cell and tissue culture, and molecular biology, as well as protein, oligonucleotide, and polynucleotide chemistry and hybridization) are ones that are well-known and commonly used in the art. Standard techniques may be used, for example, for nucleic acid purification and preparation, chemical analysis, recombinant nucleic acid, and oligonucleotide synthesis. Enzymatic reactions and purification techniques may be performed as described herein, according to manufacturer's specifications, as commonly accomplished in the art, or a combination thereof. Various techniques and procedures described herein are generally performed according to conventional methods well-known in the art and as described in various general and more specific references that are cited and discussed throughout the instant specification. See, e.g., Joseph Sambrook, David W. Russell, Molecular Cloning: A Laboratory Manual (Cold Spring Harbor Laboratory Press, 3rd ed. 2001).

The term “barcode” may refer to a label, or identifier, that conveys or is capable of conveying information (e.g., information about an analyte in a sample, a bead, a feature, a capture probe, and/or a nucleic acid barcode molecule). A barcode can be part of an analyte, a capture probe, a reporter oligonucleotide, an analyte capture agent, or nucleic acid barcode molecule, or independent of an analyte, a capture probe, a reporter oligonucleotide, an analyte capture agent, or nucleic acid barcode molecule. A barcode can be attached to an analyte, a capture probe, a reporter oligonucleotide, an analyte capture agent, or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

The term “barcoded nucleic acid molecule” generally refers to a nucleic acid molecule that results from, for example, the processing of a nucleic acid barcode molecule (e.g., a capture probe comprising a spatial barcode sequence) with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. For example, hybridization and reverse transcription of a nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may serve as a template, such as a template polynucleotide, that can be further processed (e.g., amplified) and sequenced to obtain the target nucleic acid sequence. For example, a barcoded nucleic acid molecule may be further processed (e.g., amplified) and sequenced to obtain the nucleic acid sequence of the mRNA.

In some embodiments, where nucleic acid barcode molecule comprises a single cell barcode sequence, the nucleic acid barcode molecule may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. The barcoded nucleic acid molecule may serve as a template, such as a template polynucleotide, that can be further processed (e.g., amplified) and sequenced to obtain the target nucleic acid sequence. For example, the barcoded nucleic acid molecule may be further processed (e.g., amplified) and sequenced to obtain the nucleic acid sequence of the nucleic acid molecule (e.g., mRNA).

The term “cell barcode,” as used herein, refers to a known nucleotide sequence that serves as a unique identifier for a single cell or organelle (e.g., nucleus) of a cell. A barcode can be associated with reads from a single cell or organelle (e.g., nucleus) of the cell.

A “nucleotide,” as used herein, comprises a nucleoside and a phosphate group. A “nucleoside,” as used herein, comprises a nucleobase and a five-carbon sugar (e.g., ribose, deoxyribose, or analogs thereof). When the nucleobase is bonded to ribose, the nucleoside may be referred to as a ribonucleoside. When the nucleobase is bonded to deoxyribose, the nucleoside may be referred to as a deoxyribonucleoside. A “nucleobase,” which may be also referred to as a “nitrogenous base,” can take the form of one of five types: adenine (A), guanine (G), thymine (T), uracil (U), and cytosine (C).

A “polynucleotide,” “nucleic acid,” or “oligonucleotide” refers to a linear polymer of nucleotides (or nucleosides joined by internucleosidic linkages). Generally, a polynucleotide comprises at least three nucleotides. Generally, an oligonucleotide is comprised of nucleotides that range in number from a few nucleotides (or monomeric units) to several hundreds of nucleotides (monomeric units). Whenever a polynucleotide such as an oligonucleotide is represented by a sequence of letters, such as “ATGCCTG,” it will be understood that the nucleotides are in 5′→3′ order or direction from left to right and that “A” denotes adenine, “C” cytosine, “G” denotes guanine, and “T” denotes thymine, unless otherwise noted. The letters A, C, G, and T may be used to refer to the nucleobases themselves, as described above, the nucleosides that include those nucleobases, or the nucleotides that include those bases, as is standard in the art.

Deoxyribonucleic acid (DNA) is a chain of nucleotides consisting of 4 types of nucleotides: adenine (A), thymine (T), cytosine (C), and guanine (G). Ribonucleic acid (RNA) is comprised of 4 types of nucleotides: A, C, G, and uracil (U). Certain pairs of nucleotides specifically bind to one another in a complementary fashion, which may be referred to as complementary base pairing. For example, C pairs with G and A pairs with T. In the case of RNA, however, A pairs with U. When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” “fragment sequence,” or “nucleic acid sequencing read” denotes any information or data that is indicative of the order of the nucleotide bases (e.g., A, C, G, T/U) in a molecule (e.g., whole genome, whole transcriptome, exome, oligonucleotide, polynucleotide, fragment, etc.) of DNA or RNA. It should be understood that the embodiments described herein contemplate that this sequence information may be obtained using any of the available varieties of techniques, platforms, or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic-based systems, etc., or a combination thereof.

The term “sample,” as used herein, generally refers to a biological sample of a subject. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from a group consisting of blood, plasma, serum, urine, saliva, mucosal excretions, sputum, stool and tears.

The term “primer,” as used herein generally refers to a strand of RNA or DNA that serves as a starting point for nucleic acid (e.g., DNA) synthesis. A primer may be used in a primer extension reaction, which may be a nucleic acid amplification reaction, such as, for example, polymerase chain reaction (PCR) or reverse transcription PCR (RT-PCR). The primer may have a sequence that is capable of coupling to a nucleic acid molecule. Such sequence may be complementary to the nucleic acid molecule, such as a poly-T sequence or a predetermined sequence, or a sequence that is otherwise capable of coupling (e.g., hybridizing) to the nucleic acid molecule, such as a universal primer.

As used herein, the term “cell” is used interchangeably with the term “biological cell.” Non-limiting examples of biological cells include eukaryotic cells, plant cells, animal cells, such as mammalian cells, reptilian cells, avian cells, fish cells or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells and the like. A mammalian cell can be, for example, from a human, mouse, rat, horse, goat, sheep, cow, primate or the like.

As used herein, a genome is the genetic material of a cell or organism, including animals, such as mammals, e.g., humans. In humans, the genome includes the total DNA, such as, for example, genes, noncoding DNA and mitochondrial DNA. The human genome typically contains 23 pairs of linear chromosomes: 22 pairs of autosomal chromosomes plus the sex-determining X and Y chromosomes. The 23 pairs of chromosomes include one copy from each parent. The DNA that makes up the chromosomes is referred to as chromosomal DNA and is present in the nucleus of human cells (nuclear DNA). Mitochondrial DNA is located in mitochondria as a circular chromosome, is inherited from only the female parent, and is often referred to as the mitochondrial genome as compared to the nuclear genome of DNA located in the nucleus.

The phrase “sequencing” refers to any technique known in the art that allows the identification of consecutive nucleotides of at least part of a nucleic acid. Non-limiting exemplary sequencing techniques include RNA-seq (also known as whole transcriptome sequencing), Illumina™ sequencing, direct sequencing, random shotgun sequencing, Sanger dideoxy termination sequencing, whole-genome sequencing, massively parallel signature sequencing (MPSS), sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and any combination thereof.

In general, the methods and systems described herein accomplish sequencing of nucleic acid molecules including, but not limited to, DNA (e.g., genomic DNA), RNA (e.g., mRNA, including full-length mRNA transcripts, and small RNAs, such as miRNA, tRNA, and rRNA), and cDNA. In various embodiments, the methods and systems described herein accomplish genomic sequencing of nucleic acid molecules (e.g., DNA, RNA, and mRNA). In various embodiments, the methods and systems described herein accomplish genomic sequencing of immune cell receptor sequences (e.g., DNA, RNA, and mRNA). In various embodiments, the methods and systems described herein can accomplish transcriptome sequencing, e.g., whole transcriptome sequencing of mRNA encoding immune cell receptors. In some embodiments, the methods and systems described herein can also accomplish targeted genomic sequencing of nucleic acid molecules (e.g., DNA, RNA, and mRNA). In various embodiments, the methods and systems described herein accomplish single cell genomic sequencing, for example, single cell genomic sequencing of nucleic acid molecules (e.g., RNA and mRNA) encoding immune cell receptors of single cells, such as B cell receptors (BCRs) and T cell receptors (TCRs), and/or spatial genomic sequencing.

In various embodiments, the methods and systems described herein can include high-throughput sequencing technologies, e.g., high-throughput DNA and RNA sequencing technologies. In various embodiments, the methods and systems described herein can include high-throughput, higher accuracy short-read DNA and RNA sequencing technologies. In various embodiments, the methods and systems described herein can include long-read RNA sequencing, e.g., by sequencing cDNA transcripts in their entirety without assembly. In various embodiments, the methods and systems described herein can also, for example, segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long-range molecular context is not only preserved through a sequencing process but is also preserved through the targeted enrichment process used in targeted sequencing approaches.

In general, the methods and systems described herein are directed to single cell analysis (including single- and multi-modal analyses) of genomic sequencing of nucleic acids (e.g., RNA and mRNA) encoding immune cell receptors of single cells, such as BCRs and TCRs. Single cell analysis, including single cell multi-modal analyses (e.g., single cell immune cell receptor sequencing combined with, for example, gene expression, protein expression, and/or antigen capture technologies), as well as processing and sequencing of nucleic acids, in accordance with the methods and systems described in the present application are described in further detail, for example, in U.S. Pat. Nos. 9,689,024; 9,701,998; 10,011,872; 10,221,442; 10,337,061; 10,550,429; 10,273,541; and U.S. Pat. Pub. 20180105808, which are all herein incorporated by reference in their entirety for all purposes and in particular for all written description, figures and working examples directed to processing nucleic acids and sequencing and other characterizations of genomic material.

The phrase “next generation sequencing” (NGS), as used herein, may refer to sequencing technologies having increased throughput as compared to traditional Sanger- and capillary electrophoresis-based approaches, such that these technologies have the ability to generate hundreds of thousands of relatively small sequence reads at a time. Some examples of next NGS techniques include, but are not limited to, sequencing by synthesis, sequencing by ligation, and sequencing by hybridization. An NGS protocol may be implemented using any number of or combination of sequencing technologies and devices including, for example, without limitation, the sequencing technologies provided by Illumina®, Pacific Biosciences (PacBio®), Oxford Nanopore Technologies (ONT), etc.

The term “B cells”, also known as B lymphocytes, refer to a type of white blood cell of the small lymphocyte subtype. They function in the humoral immunity component of the adaptive immune system by expressing and/or secreting antibodies. Additionally, B cells present antigens (they are also classified as professional antigen-presenting cells (APCs)) and secrete cytokines. In mammals, B cells mature in the bone marrow, which is at the core of most bones. In birds, B cells mature in the bursa of Fabricius, an immune organ where they were first discovered by Chang and Glick, (B for bursa) and not from bone marrow as commonly believed. B cells, unlike the other two classes of lymphocytes, T cells and natural killer cells, have the ability to secrete antibodies. Further, B cells can recognize intact antigen and therefore do not require that these antigens be located on or bound to peptide Major Histocompatibility Complex (pMHC) or human leukocyte antigen (HLA) molecules. BCRs allow a B cell to bind to specific antigens, against which it will initiate an antibody response.

The term “T cell”, also known as T lymphocytes, refer to a type of an adaptive immune cell. T cells develops in the thymus gland, hence the name T cell, and play a central role in the immune response of the body. T cells can be distinguished from other lymphocytes by the presence of a TCR on the cell surface. These immune cells originate as precursor cells, derived from bone marrow, and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the TCR chain, T cells can also include T cells that express αβ TCR chains, T cells that express γδ TCR chains, as well as unique TCR co-expressors (i.e., hybrid αβ-γδ T cells) that co-express the αβ and γδ TCR chains.

T cells can also include engineered T cells that can attack specific cancer cells. A patient's T cells can be collected and genetically engineered to produce chimeric antigen receptors (CAR). These engineered T cells are called CAR T cells, which forms the basis of the developing technology called CAR-T therapy. These engineered CAR T cells are grown by the billions in the laboratory and then infused into a patient's body, where the cells are designed to multiply and recognize the cancer cells that express the specific protein. This technology, also called adoptive cell transfer is emerging as a potential next-generation immunotherapy treatment.

T cells, such as the killer T cells can directly kill cells that have already been infected by a foreign invader. T cells can also use cytokines as messenger molecules to send chemical instructions to the rest of the immune system to ramp up its response. Activating T cells against cancer cells is the basis behind checkpoint inhibitors, a relatively new class of immunotherapy drugs that have recently been approved to treat lung cancer, melanoma, and other difficult cancers. Cancer cells often evade patrolling T cells by sending signals that make them seem harmless. Checkpoint inhibitors disrupt those signals and prompt the T cells to attack the cancer cells.

The term “naïve”, as used herein, can refer to B-lymphocytes or T-lymphocytes that have not yet reacted with an epitope of an antigen or that have a cellular phenotype consistent with that of a lymphocyte that has not yet responded to antigen-specific activation after clonal licensing.

The term “Fab”, also referred to as an antigen-binding fragment, refers to the variable portions of an antibody molecule with a paratope that enables the binding of a given epitope of a cognate antigen. The amino acid and nucleotide sequences of the Fab portion of antibody molecules are hypervariable. This is in contrast to the “Fc” or crystallizable fragment, which is relatively constant and encodes the isotype for a given antibody; this region can also confer additional functional capacity through processes such as antibody-dependent complement deposition, cellular cytotoxicity, cellular trogocytosis, and cellular phagocytosis.

The term “clonotype,” as used herein, refers to a set of adaptive immune cells that are clonal progeny of a fully recombined, unmutated common ancestor. T cell clonotypes are generally distinguished by the nucleotide sequence of the rearranged TCR, which does not undergo somatic hypermutation (SHM) in most vertebrate species. B cell clonotypes are commonly divergent from each other at the nucleotide level. For this reason, B cell clonotypes also frequently contain multiple exact subclonotypes.

For example, to understand what constitutes members of a clonotype, one can start with the original progenitor cell for a given lineage of B cells or T cells, this progenitor cell commonly referred to as the parent clone, which is a single cell to which all daughter cells will be genetically related, though their B or T cell receptors and exact antigen specificity may differ and diverge over time. Collectively, this parent clone and all its daughter cells constitute a clonotype (or clonotype group). As stated above, accurate identification of the members of a clonotype may be critical not just from a biological perspective, but also from the biomedical perspective, as correct identification of all of the members of a given clonotype can be useful in the design of vaccines (e.g., which antibody lineages can be expanded by a vaccine or are expanded successfully or unsuccessfully by a vaccine), in the monitoring of B cell-mediated immune disease (e.g., myasthenia gravis, lupus, B cell lymphoma), and in other settings (what antibodies are found in the tumor microenvironment or other immune niches during clinical disease).

Clonotyping may be performed and/or visualized using any of the systems and/or processes described in PCT/US2021/019120, as well as U.S. application Ser. Nos. 17/233,029 and 17/182,147, which are incorporated herein by reference in their entirety.

The term “exact subclonotype,” as used herein, refers to a subset of cells within a clonotype that share identical immune receptor sequences at the nucleotide level, spanning the entirety of the V, D, and J genes and the V(D)J junction. Exact subclonotypes share the same V, D, J, and C gene annotations (e.g. cells that have identical V(D)J sequences but different C genes or isotypes are split into distinct exact subclonotypes).

The phrase “clonal selection” refers to the selection and activation of specific B lymphocytes and T lymphocytes by the binding of epitopes to B cell receptors or T cell receptors with a corresponding fit and the subsequent elimination (negative selection) or licensing for clonal expansion (positive selection) of a B or T lymphocyte after binding of an antigenic determinant.

The phrase “clonal expansion” refers to the proliferation of B lymphocytes and T lymphocytes activated by clonal selection in order to produce a clonal population of daughter cells with the same antigen specificity and functional capacity. In the case of T lymphocytes this antigen specificity is exact at the nucleotide and protein level and in the case of B lymphocytes this antigen specificity can be exact at the nucleotide and protein level or mutated relative to the parent population by mutations at the nucleotide level (and by extension the protein level). This enables the body to have enough antigen-specific lymphocytes to mount an effective immune response.

The term “cytokines” refers to a wide variety of intercellular regulatory proteins produced by many different cells in the body, which ultimately control every aspect of body defense. Cytokines activate and deactivate phagocytes and immune defense cells, enhance or inhibit the functions of the different immune defense cells, and promote or inhibit a variety of nonspecific body defenses.

The phrase “T helper lymphocytes”, also referred to as helper cells, refer to a type of white blood cell that orchestrate the immune response and enhance the activities of the killer T-cells (those that destroy pathogens) and B cells (antibody and immunoglobulin producers).

The phrase “affinity maturation” refers to the gradual modification of the paratope and entire B cell receptor as a result of somatic hypermutation. B lymphocytes with higher affinity B cell receptors that can bind the epitope more tightly and, therefore, bind the epitope for a longer period are able to proliferate more and survive longer. These B cells can eventually differentiate into plasma cells, which secrete their antibodies and form the basis of serum-mediated immunity.

The phrase “somatic hypermutation” (SHM) refers to a cellular mechanism by which the adaptive immune system adapts to foreign elements confronting it (e.g. viruses, bacteria, biomolecules). A major component of the process of affinity maturation, SHM diversifies B cell receptors used to recognize foreign elements (antigens) and allows the immune system to adapt its response to new threats during the lifetime of an organism. Somatic hypermutation involves a programmed process of mutation predominantly affecting select framework and complementarity-determining regions of immunoglobulin genes. Unlike germline mutation, SHM operates at the level of an organism's individual immune cells. These mutations are not transmitted to the organism's offspring but are transmitted to daughter cells of individual B cell clones. Mistargeted somatic hypermutation is a likely mechanism in the development of B cell lymphomas and many other cancers. Somatic hypermutation can also lead to the acquisition of non-VDJ template DNA within B cell receptor sequences, such as LAIR1 insertions in malaria-specific neutralizing antibodies.

Somatic hypermutation is a distinct diversification mechanism from isotype switching (also called class switching). Mutations acquired during somatic hypermutation eventually lead to isotype switching, in which a B cell's antibody can be coupled to different functions by switching to a different Fc/constant region sequence. Isotype switching is an irreversible process, in that once a B cell has switched from a given constant region (e.g. IGHM) to a new constant region (e.g. IGHA1) it can no longer use the IgM constant region as the DNA encoding the IgM Fc is excised and removed during isotype switching.

The term “contig”, originating from the term “contiguous”, refers to a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequences and to overlapping physical segments (fragments) contained in clones depending on the context. Note that clone, in reference to overlapping clones, refers to individual bacteria or constructs (e.g. phagemids, cosmids, etc.) containing distinct insertions of genomes that were utilized in early efforts to map genomes

The phrase “heavy chain” refers to the large polypeptide subunit of an antibody (immunoglobulin). The first recombination event to occur is between one D and one J gene segment of the heavy chain locus. Any DNA between these two gene segments is deleted. This D-J recombination is followed by the joining of one V gene segment, from a region upstream of the newly formed DJ complex, forming a rearranged VDJ gene segment. All other gene segments between V and D segments are now deleted from the cell's genome. Primary transcript (unspliced RNA) is generated containing the VDJ region of the heavy chain and both the constant mu and delta chains (Cμ and Cδ) (i.e., the primary transcript contains the segments: V-D-J-Cμ-Cδ). The primary RNA is processed to add a polyadenylated (poly-A) tail after the Cμ chain and to remove sequence between the VDJ segment and this constant gene segment. Translation of this mRNA leads to the production of the IgM heavy chain protein and the IgD heavy chain protein (its splice variant). Expression of the immunoglobulin heavy chain with one or more surrogate light chains constitutes the pre-B cell receptor that allows a B cell to undergo selection and maturation.

The phrase “light chain” refers to the small polypeptide subunit of an antibody (immunoglobulin). The kappa (κ) and lambda (λ) chains of the immunoglobulin light chain loci rearrange in a very similar way, except that the light chains lack a D segment in other words, the first step of recombination for the light chains involves the joining of the V and J chains to give a VJ complex before the addition of the constant chain gene during primary transcription. Translation of the spliced mRNA for either the kappa or lambda chains results in formation of the Ig κ or Ig λ light chain protein. Assembly of the Ig μ heavy chain and one of the light chains results in the formation of membrane bound form of the immunoglobulin IgM that is expressed on the surface of the immature B cell. B cells may express up to two heavy chains and/or two light chains in respectively rare and uncommon instances through a phenomenon known as allelic inclusion. This phenomenon can only be directly observed using single-cell technologies, though it can be inferred with a degree of uncertainty using a combination of bulk sequencing technologies and probabilistic inference via an extension of the birthday paradox.

The phrase “complementarity-determining regions” (CDRs) refers to part of the variable chains in immunoglobulins (antibodies) and T cell receptors, generated by B cells and T cells respectively, where these molecules are particularly hypervariable. The antigen-binding site of most antibodies and T cell receptors is typically distributed across these CDRs, collectively forming a paratope. However, there are many documented examples of paratopes that enable antigen recognition that fall outside of the CDRs. As the most variable parts of the molecules, CDRs are crucial to the diversity of antigen specificities and immune cell receptor sequences generated by lymphocytes.

V(D)J recombination is a genetic recombination mechanism that occurs in developing lymphocytes during the early stages of T and B cell maturation. Through somatic recombination, this mechanism produces a highly diverse repertoire of antibodies/immunoglobulins and T cell receptors (TCRs) found in B cells and T cells, respectively. This process is a defining feature of the adaptive immune system and these receptors are defining features of adaptive immune cells.

V(D)J recombination occurs in the primary immune organs (bone marrow for B cells and thymus for T cells) and in a generally random fashion. The process leads to the rearranging of variable (V), joining (J), and in some cases, diversity (D) gene segments. As discussed above, the heavy chain possesses numerous V, D, and J gene segments, while the light chain possesses only V and J gene segments. The process ultimately results in novel amino acid sequences in the antigen-binding regions of immunoglobulins and TCRs that allow for the recognition of antigens from nearly all pathogens including, for example, bacteria, viruses, and parasites. Furthermore, the recognition can also be allergic in nature or may recognize host tissues and lead to autoimmunity.

Human antibody molecules, including B cell receptors (BCRs), include both heavy and light chains, each of which contains both constant (C) and variable (V) regions, and are genetically encoded on three loci. The first is the immunoglobulin heavy locus on chromosome 14, containing the gene segments for the immunoglobulin heavy chain. The second is the immunoglobulin kappa (κ) locus on chromosome 2, containing the gene segments for part of the immunoglobulin light chain. The third is the immunoglobulin lambda (λ) locus on chromosome 22, containing the gene segments for the remainder of the immunoglobulin light chain.

Each heavy or light chain contains multiple copies of different types of gene segments for the variable regions of the antibody proteins. For example, the human immunoglobulin heavy chain region contains nine C gene segments (Cα1, Cα1, Cγ1, Cγ2, Cγ3, Cγ4, Cδ, Cε, and Cμ), 44 V gene segments, 27 D gene segments and 6 J gene segments. The number of given segments present in any individual can vary, as these gene segments are carried in haplotypes; for this reason, inference of both the alleles present within an individual and the germline sequence of those alleles is an important step in correctly identifying B cell clonotypes. The light chains possess two C gene segments (Cλ and Cκ) and numerous V and J gene segments, but do not have D gene segments. DNA rearrangement causes one copy of each type of gene segment to mate with any given lymphocyte, generating a substantial antibody repertoire. Approximately 10¹⁴ combinations are possible, with 1.5×10² to 3×10³ potentially removed via self-reactivity.

Accordingly, each naïve B cell makes an antibody with a unique Fab site through a series of gene recombination steps, and later mutations, with the specific molecules of the given antibody attaching to the B cell's surface as a B cell receptor (BCR). These BCRs are then available to react with epitopes of an antigen.

The term “CDR3 (Complementarity-Determining Region 3), as used herein, refers to the third complementarity determining region which is a portion of the amino acid sequence of a T or B cell receptor. The nucleotide region encoding CDR3 spans the V(D)J junction, making it more diverse than that of the other CDRs. This serves as a useful way to identify unique chains.

When the immune system encounters an antigen, epitopes of that antigen will be presented to many B lymphocytes. B lymphocytes must first rearrange a heavy chain that enables pre-B cell receptor ligand binding. B lymphocytes that bind multivalent self-targets after rearrangement of the light chain too strongly are eliminated and die or undergo a secondary recombination event, while B cells that do not bind self-targets too strongly are licensed to exit the bone marrow. The latter becomes available to respond to non-self antigens and to undergo clonal expansion. This process is known as clonal selection.

Cytokines produced by activated CD4 T helper lymphocytes enable those activated B lymphocytes (B cells) to rapidly proliferate to produce large clones of thousands of identical B cells. More specifically, when under threat (i.e., via bacteria, virus, etc.), the body releases white blood cells by the immune system. CD4 T lymphocytes help the response to a threat by triggering the maturation of other types of white blood cell. They produce special proteins, called cytokines, have plural functions, including the ability to summon all of the other immune cells to the area, and also the ability to cause nearby cells to differentiate (become specialized) into mature B cells and T cells.

Accordingly, while only a few B cells in the body may have an antibody molecule that can bind a particular epitope, eventually many thousands of cells are produced with the right specificity, allowing the body's immune system to act en masse. This is referred to as clonal expansion. Natural phenomena such as IgA deficiency and murine transgenic models have shown that there are multiple paths by which a B cell receptor can acquire novel antigen specificity even from a very limited repertoire through the processes of somatic hypermutation and affinity maturation.

As the B cells proliferate, they undergo affinity maturation as a result of somatic hypermutation. This allows the B cells to “fine-tune” the paratopes of the antibody to more effectively fit with the recognized epitopes. B cells with high affinity B cell receptors on their surface bind epitopes more tightly and for a longer period of time, which enables these cells to selectively proliferate. Over the course of this proliferation and expansion, these variant B cells differentiate into plasma cells that synthesize and secrete vast quantities of antibodies with Fab sites that fit the target epitopes very precisely.

The phrase “immune cell” refers to a cell that is part of the immune system and that helps the body fight infections and other diseases. Immune cells include innate immune cells (such as basophils, dendritic cells, neutrophils, etc.) that are the first line of body's defense and are deployed to help attack the invading foreign cells (e.g., cancer cells) and pathogens. The innate immune cells can quickly respond to foreign cells and pathogens to fight infection, battle a virus, or defend the body against bacteria. Immune cells can also include adaptive immune cells (such as lymphocytes including B cells and T cells). The adaptive immune cells can come into action when an invading foreign cells or pathogens slip through the first line of body's defense mechanism. The adaptive immune cells can take longer to develop, because their behaviors evolve from learned experiences, but they can tend to live longer than innate immune cells. Adaptive immune cells remember foreign invaders after their first encounter and fight them off the next time they enter the body. Both types of immune cells employ important natural defenses in helping the body fight foreign cells and pathogens for fighting infections and other diseases.

Accordingly, the immune cells of the disclosure can include, but are not limited to, neutrophils, eosinophils, basophils, mast cells, monocytes, macrophages, dendritic cells, natural killer cells, and lymphocytes (such as B cells and T cells). The immune cells of the disclosure can further include dual expresser cells or DE (such as unique dual-receptor-expressing lymphocytes that co-express functional B cell receptor (BCR) and T cell receptor (TCR)), cells with adaptive immune receptors that may diversify or may not diversify (including immune cells expressing a chimeric antigen receptor with a fixed nucleotide sequence or with the capacity to mutate), and TCR co-expressors (i.e., hybrid αβ-γδ T cells) that co-express both and γδ TCR chains.

The phrase “immune cell receptor”, “immune receptor”, or “immunologic receptor” refers to a receptor or immune cell receptor sequence, usually on a cell membrane, which can recognize components of pathogenic microorganisms (e.g., components of bacterial cell wall, bacterial flagella or viral nucleic acids) and foreign cells (e.g., cancer cells), which are foreign and not found naturally on the host cells, or binds to a target molecule (for example, a cytokine), and causes a response in the immune system. The immune cell receptors of the immune system can include, but are not limited to, pattern recognition receptors (PRRs), Toll-like receptors (TLRs), killer activated and killer inhibitor receptors (KARs and KIRs), complement receptors, Fc receptors, B cell receptors, and T cell receptors.

The phrase “immune cell receptor sequences” of an immune cell receptor include both heavy and light chains, each of which contains both constant (C) and variable (V) regions. For example, B cell receptors (BCRs) or B cell receptor sequences (including human antibody molecules) comprise of immunoglobulin heavy and light chains, each of which contains both constant (C) and variable (V) regions. Each heavy or light chain not only contains multiple copies of different types of gene segments for the variable regions of the antibody proteins, but also contains constant regions. For example, the BCR or human immunoglobulin heavy chain contains 9 constant gene segments, 44 Variable (V) gene segments, 27 Diversity (D) gene segments, and 6 Joining (J) gene segments. The BCR light chains also possess 2 constant gene segments and numerous V and J gene segments, but do not have any D gene segments. DNA rearrangement (i.e., recombination events) in developing B cells can cause one copy of each type of gene segment to go in any given lymphocyte, generating an enormous antibody repertoire. Accordingly, the primary transcript (unspliced RNA) of a BCR heavy chain can be generated containing the VDJ region of the heavy chain and both the constant mu and delta chains (Cμ and Cδ), i.e., the heavy chain primary transcript can contain the segments: V-D-J-Cμ-Cδ). In case of the B cell receptor and human immunoglobulin light chain, the first step of recombination for the light chains involves the joining of the V and J chains to give a VJ complex before the addition of the constant chain gene during primary transcription. Translation of the spliced mRNA for either the constant κ (Cκ) or λ (Cλ) chains results in formation of the Ig κ or Igλ light chain protein.

In general, most T cell receptors (TCR) are composed of an alpha (α) chain and a beta (β) chain, each of which contains both constant (C) and variable (V) regions. Thus, the most common type of a T cell receptor is called an alpha-beta TCR because it is composed of two different chains, one α-chain and one beta β-chain. A less common type of TCR is the gamma-delta TCR, which contains a different set of chains, one gamma (γ) chain and one delta (δ) chain. The T cell receptor genes are similar to immunoglobulin genes for the BCR and undergo similar DNA rearrangement (i.e., recombination events) in developing T cells as for the B cells. For example, the alpha-beta TCR genes also contain multiple V, D, and J gene segments in their beta chains and V and J gene segments in their alpha chains, which are re-arranged during the development of the T cells to provide a cell with a unique T cell antigen receptor. Thus, the β-chain of the TCR can contain Vβ-Dβ-Jβ gene segments and constant domain (Cβ) genes resulting in a Vβ-Dβ-Jβ-Cβ sequence of the TCR β-chain. The re-arrangement of the alpha (α) chain of the TCR follows (3 chain rearrangement, and can include Vα-Jα gene segments and constant domain (Cα) genes resulting in a Vα-J α-Cα sequence of the TCR α-chain. Similar to the alpha-beta TCRs, the TCR-γ chain is produced by V-J recombination and can contain Vγ-Jγ gene segments and constant domain (Cγ) genes resulting in a Vγ-Jγ-Cγ sequence of the TCR γ-chain, while the TCR-δchain is produced using V-D-J recombination, and can contain Vδ-Dδ-Jδ gene segments and constant domain (Cδ) genes resulting in a Vδ-Dδ-Jδ-Cδ sequence of the TCR δ-chain.

The phrase “immune cell receptor constant region sequence” or “immune receptor constant region sequence” refers to the constant region or constant region sequence of an immune cell receptor. For example, the immune cell receptor constant region sequence or immune receptor constant region sequence can include, but is not limited to, the constant mu (Cμ) and delta (Cδ) region genes and sequences of a BCR and immunoglobulin heavy chain, the constant lambda (Cλ) and kappa (Cκ) region genes and sequences of a BCR and immunoglobulin light chain, the alpha constant (Cα) region genes and sequences of a TCR α-chain sequence, the beta constant (Cβ) region genes and sequences of a TCR β-chain sequence, the gamma constant (Cγ) region genes and sequences of a TCR γ-chain sequence, and the delta constant (Cδ) region genes and sequences of a TCR δ-chain sequence.

With this understanding of the immune cell's purpose in fighting off attacking foreign antigens, the pharmaceutical industry has strongly focused on designing vaccines with the ability to expand antibody lineages directed towards specific B cells with shared antigen specificity. To most effectively determine the efficacy of a vaccine or antitumor antibody therapy, it is essential to be able to accurately identify cell members of a clonotype, which potentially share common or similar BCRs or antigen specificity. The pharmaceutical industry has also directed its efforts to isolate antibodies and antibody lineages against non-foreign targets for the purpose of developing antibody-based therapeutics for a broad array of disease states including autoimmune disease (anti-inflammatory targets), cancer (checkpoint inhibitors and other targets), and other conditions such as osteoporosis. Similarly, knowing the fine specificities of different antibody lineages elicited by a vaccine may be essential to understanding serum neutralization profiles and global epitope maps of an entire virus. This same concept applies to understanding how a patient's adaptive immune system can render drugs (e.g., adalimumab) ineffective through the emergence of anti-drug antibodies and distinct anti-drug antibody lineage.

### III. Barcoding and Sequencing Methodologies

Various barcoding and sequencing methodologies (e.g., single cell technologies or combinations of single cell technologies) may be used to generate data that provides sequence information for individual cells. Such methodologies (e.g., single cell technologies) include, but are not limited to, non-droplet single cell sequencing technologies, droplet-based microfluidic single cell sequencing technologies, microwell array-based and nanowell array-based single cell sequencing technologies, in situ sequencing technologies, and spatial analysis methodologies, e.g., spatially indexed single cell sequencing technologies, which are further described herein.

Any known sequencing methods (e.g., including single cell sequencing methods and spatial analysis methodologies) can be used to provide immune cell data (e.g., single immune cell sequencing data or spatial data) in various embodiments. In various embodiments, with single cell sequencing methods, single cells can be separated into partitions such as droplets or wells, wherein each partition comprises a single cell with a known identifier like a barcode. The barcode can be attached to a support, for example, a bead, such as a solid bead or a gel bead.

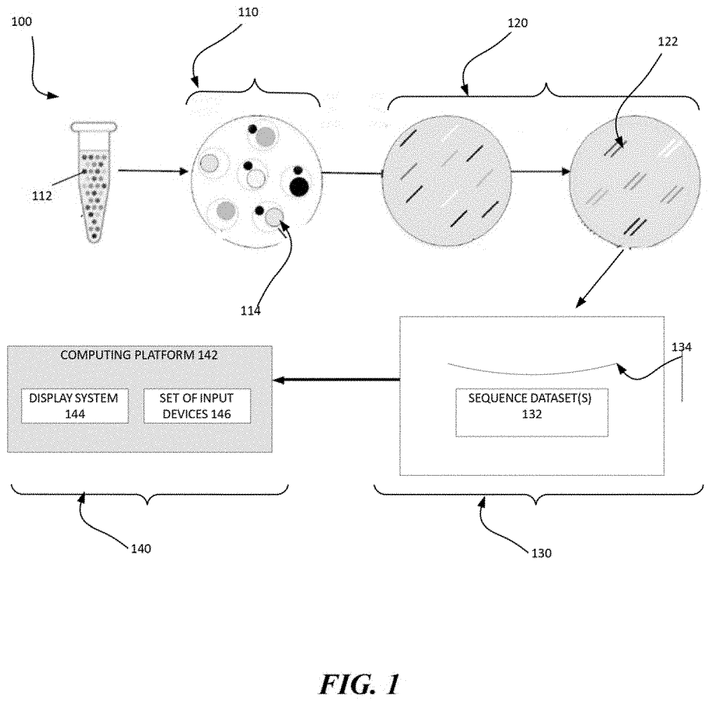

FIG. 1 is a schematic diagram of a workflow 100 for single cell sequencing in accordance with various embodiments. Workflow 100 in FIG. 1 is an example of one manner in which single cell sequencing (e.g., single cell or spatial analysis methodologies) may be implemented. It should be understood that in other example embodiments, workflow 100 may include one or more features in addition to or in place of the features described herein, one or more fewer features than described herein, or a combination thereof. Workflow 100 may be used to generate sequence information for individual cells such as, for example, individual immune cells. For example, workflow 100 may be used to generate sequence information, antigen binding information, or a combination thereof, which may be used for identifying V(D)J information, clonotype information, antigen specificity, one or more other types of information, or a combination thereof in accordance with various embodiments. Workflow 100 includes sample preparation and processing 110, library construction 120, sequencing 130, and data analysis 140, which are further described below. The workflow provided in FIG. 1 may include using, for example, single cell sequencing methodologies or spatial analysis methodologies. Accordingly, the single cell methodologies described below with respect to FIG. 1 are merely examples of how the workflow in FIG. 1 may be implemented and are not meant to limiting. Spatial analysis methodologies that may be included in the workflow in FIG. 1 are described further below.

Sample preparation and processing 110, library construction 120, sequencing 130, and data analysis 140 may be performed using any number of or combination of the methodologies, systems, or concepts described herein and/or any number of or combination of methodologies, systems, or concepts described in U.S. Pat. Nos. 10,323,278, 10,550,429, 10,815,525, 10,725,027, 10,343,166, 10,583,440; U.S. Patent Application Publication Nos. 2018/0105808, 2018/0179590, 2019/0367969; U.S. Provisional Patent Application Nos. 63/135,493, 63/135,504, 63/135,514, and 63/135,519; and International Publication No. WO 2019/040637, each of which is incorporated herein by reference in its entirety.

Sample Preparation and Processing

In one or more embodiments, sample preparation and processing 110 includes the preparation and processing of sample 112 comprised of cells. Sample preparation/processing 110 can include, without limitation, partition-based approaches for processing single cells or their components, or spatial array based methodologies, described further herein. In various embodiments, sample preparation and processing 110 includes labeling sample 112 with tags 113. For example, sample may be labeled with one or more different ones of tags 113. In one or more embodiments, at least a portion of tags 113 takes the form of oligonucleotides (or polynucleotides). An oligonucleotide may be attached to a carrier molecule (e.g., a lipid) that carries the oligonucleotide to a cell. In one or more embodiments, the carrier molecule facilitates “labeling” of the cell via insertion past the cell membrane, thereby labeling the cell with the oligonucleotide attached to the carrier molecule.

In one or more embodiments, sample 112 is comprised of immune cells (e.g., T cells, B cells, leukocytes, etc.). Tags 113 may include, for example, antibodies, fluorescent antibodies, multimers, B cell antigens, other types of markers, or a combination thereof. The multimers may include, for example, but are not limited to, dextramers (e.g., using Dextramer® technology), tetramers, pentamers, dodecamers, one or more other types of multimers, or a combination thereof. The B cell antigens may include, but are not limited to, for example, whole proteins, protein domains, peptides, virus-like particles, lipoparticles, one or more other types of antigens, or a combination thereof. In some cases, tags 113 may include multimerized forms of any one or combination of these different types antigens.

In one or more embodiments, sample preparation and processing 110 includes the preparation and processing of sample 112. For example, sample preparation and processing 110 includes, without limitation, the partitioning of sample 112 into wells (e.g., microwell arrays, nanowell arrays, etc.), droplets, or some other form of partition. Generally, in single cell sequencing, sample 112 is partitioned into a plurality of partitions 114 for a plurality of single cells.

A “partition” of the partitions 114 of sample 112 may capture a single cell. For example, a partition may generally include a single cell (or a lysate of a single cell) and a support. In one or more embodiments, this support includes (e.g., has bonded to it) a plurality of nucleic acid barcode molecules (e.g., polynucleotides such as, but not limited to, oligonucleotides, etc.) that uniquely identify the corresponding single cell. The nucleic acid barcode molecules of the plurality of nucleic acid barcode molecules share a common barcode sequence (or barcode). For example, in one or more embodiments, the support takes the form of a bead (e.g., a gel or hydrogel bead) and a plurality of oligonucleotides are provided on the bead that share a common barcode sequence. Thus, the bead is associated with a unique barcode sequence that can be repeated for the plurality of oligonucleotides on the bead. In some embodiments, these beads are gel beads. A gel bead emulsion (GEM) contains a single cell, a single gel bead, and one or more reagents (e.g., lysis reagents, reverse transcriptase enzymes or reagents, etc.). In some embodiments each partition is intended to capture a single cell.

Sample preparation and processing 110 can result in the partitioning of sample 112 into partitions 114 with at least a subset of partitions 114 containing single cells that are comprised of analytes of interest (e.g., nucleic acid molecules such as, but not limited to, mRNA, etc.). In some embodiments, sample preparation and processing 110 further includes lysing the single cells within the partitions to release cellular components, including the analytes of interest (e.g., nucleic acid molecules or components), and barcoding the cellular components. The nucleic acid components released from a single cell within a particular partition are barcoded with the common barcode sequence that is associated with the support (e.g., bead) of the partition to thereby form barcoded nucleic acid molecules. These barcoded nucleic acid molecules may be sequenced to yield different types of information about the cells from which they originated.

In one or more embodiments, a partition that includes a single cell and a support associated with a unique barcode sequence may further include one or more reagents (e.g., lysis reagents including, for example, without limitation, bioactive reagents) to allow for processing of the single cell and the release of the cellular components of the single cell via lysis. For example, lysis may be used to release nucleic acid components such as, but not limited to, RNA (e.g., mRNA), DNA, or both. Further, lysis may also cause the release of the plurality of nucleic acid barcode molecules on the support such that these nucleic acid barcode molecules may anneal to and barcode the complements of the released nucleic acid components. In this manner, the various nucleic acid components that are released all include the common barcode sequence associated with the support within that partition.

For example, the support may be a bead that can be degraded (e.g., dissolved) via one or more reagents to release the nucleic acid barcode molecules (e.g., oligonucleotides that all share identical barcode sequences). The nucleic acid barcode molecules released from the support, as well as the nucleic acid components released from the single cell (e.g., mRNA) and reagents (e.g., reverse transcription (RT) reagents), within a partition are used to perform a nucleic acid extension reaction (e.g., reverse transcription of polyadenylated mRNA) to generate barcoded nucleic acid molecules (e.g., barcoded cDNA) within the partition. For example, all cDNA molecules that trace back to a same single cell within the partition will share an identical barcode sequence. In this manner, the barcoded nucleic acid molecules generated in a partition enable future sequencing to be mapped back to the original single cells from which the barcoded nucleic acid molecules originated.

Various protocols known in the art can be employed to generate sample 112 for use with one or more of the embodiments described herein. Sample 112 (e.g., suspension) can be generated from any one or more types of cells. For example, such cells may include eukaryotic cells (e.g., eukaryotic cells with a chromatin structure). Further, cells from fresh or cryopreserved cell lines (e.g., human cell lines, mouse cell lines, etc.), as well as more fragile primary cells, may be used. In one or more embodiments, the cells in sample 112 include, but are not limited to, immune cells (e.g., B cells, T cells), peripheral blood mononuclear cells (PBMCs), bone marrow mononuclear cells (BMMCs), lymphocytes, or a combination thereof. Still further, sample 112 may be formed by cells from a single donor or multiple donors.

Sample 112 may be obtained or extracted from one or more subjects including, for example, without limitation, any one of or combination of a diseased subject, a convalescent subject, a vaccinated subject, a healthy subject, an immunosuppressed subject, a subject having an autoimmune disorder or issue, or some other type of subject. In some cases, the biological sample may be extracted from one or more subjects at a timepoint prior to a disease outbreak. In other cases, the biological sample may be extracted from a population during a disease outbreak, regardless of whether the subject or population is symptomatic or asymptomatic. A subject, who may also be referred to as a donor in some cases, may be an animal subject such as, for example, without limitation, a mammal, a bird, a reptile, an amphibian, or a fish. A mammal or mammalian subject may take the form of, for example, without limitation, a human, a swine, a monkey, an ape, a dog, a cat, a mouse, a rat, or some other type of subject.

Library Construction

Library construction 120 (based on, e.g., single cell or spatial analysis) can include the generation of a library 122 that contains a plurality of DNA fragments. These DNA fragments may be utilized for sequencing, which occurs in sequencing 130. In one or more embodiments, barcoded cDNA molecules recovered from the partitions 114 formed and processed in sample preparation and processing 110 can be used as templates for multiplexed PCR to produce a single cell library. Library 122 may include molecules from one or more samples, molecules from samples from one or more donors, molecules from multiple libraries corresponding to one or more donors, or a combination thereof.

In one or more embodiments, library construction 120 includes library preparation. Library preparation may include, for example, adding one or more adapter sequences, a sample index (SI) sequence, or a combination thereof to the recovered barcoded cDNA molecules in library construction 120. An SI sequence may include, for example, without limitation, one or more oligonucleotides (e.g., four oligonucleotides) that enable unique identification of the original sample, e.g., sample 112.

Sequencing

Sequencing 130 is performed to generate one or more sequence datasets (or sequencing datasets) 132 based on the fully constructed library 122. Sequencing 130 can generate immune cell data set 132 that provides immune cell receptor information, e.g., on a single cell basis or a spatial basis. Sequence dataset 132 may include a sequence (e.g., a codon sequence) for a molecule (e.g., barcoded cDNA molecule) included in library 122. Sequencing 130 may be performed using, for example, but is not limited to a next-generation sequencing (NGS) protocol. This NGS protocol may be implemented using any number of or combination of sequencing technologies and devices including, for example, without limitation, the sequencing technologies provided by Illumina®, Pacific Biosciences (PacBio®), Oxford Nanopore Technologies (ONT), etc.

Sequence dataset 132 may be generated in a format that is compatible with data analysis 140 described below or that can be converted into a format compatible with data analysis 140. As one non-limiting example, sequence dataset 132 may be generated in a FASTQ format, which is a text-based format for storing biological sequences such as nucleotide sequences. In other embodiments, a different file format may be used for sequence dataset 132. In one or more embodiments, sequence dataset 132 is stored in data store 134. For example, data store 134 can be configured to store sequence dataset 132 for a single cell, including data for receptors (e.g., immune cell receptors), fragments thereof, or a combination thereof from single cells. Further, various software tools can be employed for processing and sending sequence dataset 132 as input into the downstream data analysis 140 portion of workflow 100.

In one or more embodiments, sample preparation and processing 110, library construction 122, and sequencing 130 may be implemented using next generation sequencing (NGS) technology. One example of an NGS technology is Chromium single-cell RNA-sequencing technology (available from 10× Genomics). The Chromium single-cell RNA-sequencing technology takes a sample, such as sample 112, containing cells of interests (e.g., lymphoid cells such as B cells or T cells), uses microfluidic partitioning to capture single cells in sample 112, and prepares uniquely barcoded beads, which may be referred to as gel beads-in-emulsions (GEMs). These GEMS may then be used to derive one or more libraries, such as, for example, library 122. These one or more libraries may be, for example, barcoded cDNA libraries. Further, these libraries may be sequenced using, for example, Illumina® sequencing instruments to generate sequencing dataset 132. The various embodiments of the Chromium™ single-cell RNA-sequencing technology, as described herein, may include the use of any number of platforms such as, for example, without limitation, the One Sample, One GEM Well, One Flowcell platform; the One Sample, One GEM well, Multiple Flowcells platform; the One Sample, Multiple GEM Wells, One Flowcell platform; the Multiple Samples, Multiple GEM Wells, One Flowcell platform, or a combination thereof. It is understood that other sequencing technologies and platforms are also contemplated for use in generating sequence dataset 132.

Although sample preparation and processing 110, library construction 122, and sequencing 130 have been described with respect to a single sample 112, in some embodiments, these operations may be performed for multiple samples from a single subject or multiple samples from multiple subjects.

It is understood that various systems and methods with the embodiments herein are contemplated and can be employed to simultaneously analyze the inputted single cell sequencing data or spatial data for sequence analysis in accordance with various embodiments.

Spatial Analysis Methodologies

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), both of which are available at the 10× Genomics Support Documentation website, and can be used herein in any combination. Further non-limiting aspects of spatial analysis methodologies and compositions are described herein.

Array-based spatial analysis methods involve the transfer of one or more analytes from a biological sample to an array of features on a substrate, where a feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

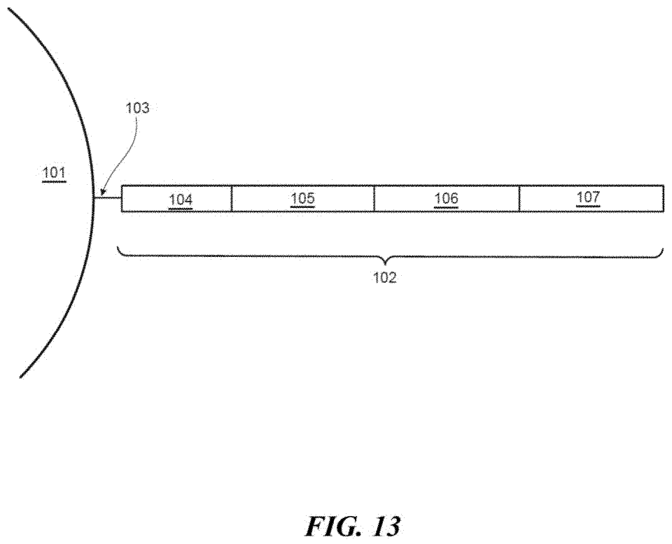

FIG. 13 is a schematic diagram showing an exemplary capture probe, as described herein. As shown in FIG. 13, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 13 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

Referring again to FIG. 13, in some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

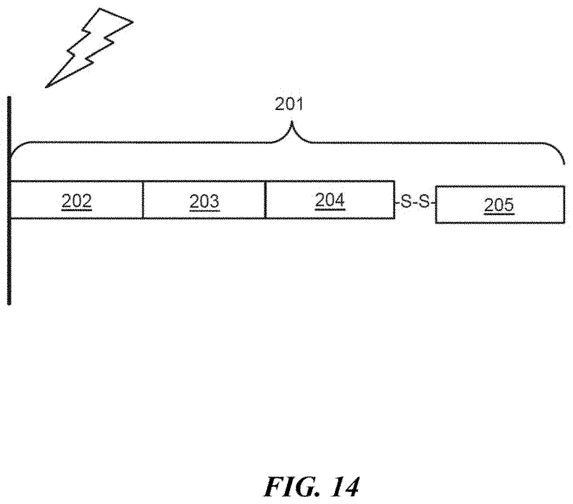

FIG. 14 is a schematic illustrating a cleavable capture probe, wherein the cleaved capture probe can enter into a non-permeabilized cell and bind to analytes within the sample. As shown in FIG. 14, the capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

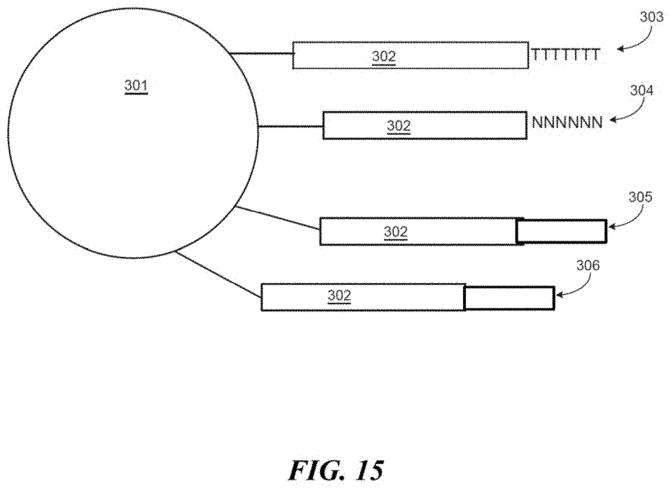

FIG. 15 is a schematic diagram of an exemplary multiplexed spatially-barcoded feature. In FIG. 15, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 15, capture-probe barcoded constructs can be tailored for analyses of any given analyte associated with a nucleic acid and capable of binding with such a construct. For example, the schemes shown in FIG. 15 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MEW multimers described herein), and a V(D)J sequence of an immune cell receptor (e.g., T-cell receptor) or antigen binding molecule (ABM).

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample (e.g., a tissue sample) with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., a tissue sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. An array feature location represents a position relative to a coordinate reference point (e.g., an array location, a fiducial marker) for the array. Accordingly, the feature location has an “address” or location in the coordinate space of the array.

Exemplary spatial methodologies for generating immune cell data (e.g., spatial datasets of at least one of immune cell receptors, antibodies, or fragments thereof from a tissue sample) are further described in WO2021247568 and WO2021247543, which are hereby incorporated by reference in their entirety. Such immune cell data may be obtained from tissue samples, e.g., tissue sections. The tissue section can be a fresh frozen tissue section, a fixed tissue section, or an FFPE tissue section. In some embodiments, the tissue sample is fixed and/or stained (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Tissue samples are also described in Section (I)(d) of WO 2020/176788 and/or U.S. Patent Application Publication No. 2020/0277663.

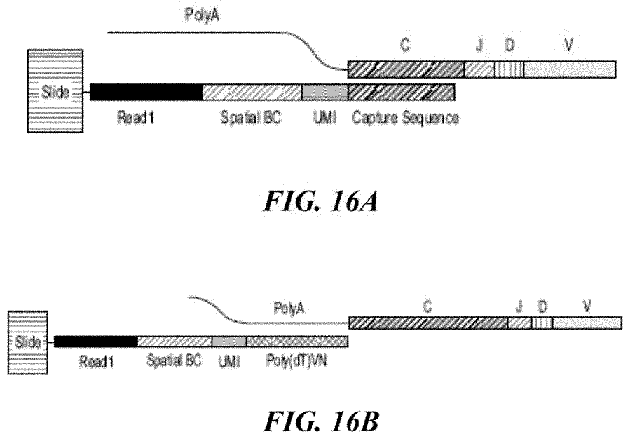

An exemplary embodiment of a spatial methodology for generating immune cell data (e.g., sequence data for an antigen binding molecule (ABM)) is depicted in FIG. 16A. An exemplary capture probe with a capture sequence that specifically binds to a nucleic acid sequence encoding a constant region of an ABM is depicted in FIG. 16A. In some embodiments, the ABM is selected from: a TCR alpha chain, a TCR beta chain, a TCR gamma chain, a TCR delta chain an immunoglobulin kappa light chain, an immunoglobulin lambda light chain, an immunoglobulin heavy chain. In some embodiments, the first capture sequence binds specifically to a nucleic acid sequence encoding a constant region of the T cell receptor alpha chain. In some embodiments, the first capture sequence binds specifically to a nucleic acid sequence encoding a constant region of the T cell receptor beta chain. In some embodiments, the first capture sequence binds specifically to a nucleic acid sequence encoding a constant region of the T cell receptor delta chain. In some embodiments, the first capture sequence binds specifically to a nucleic acid sequence encoding a constant region of the T cell receptor gamma chain. In some embodiments, the first capture sequence binds specifically to a nucleic acid sequence encoding a constant region of the immunoglobulin kappa light chain. In some embodiments, the first capture sequence binds specifically to a nucleic acid sequence encoding a constant region of the immunoglobulin lambda light chain. In some embodiments, the first capture sequence binds specifically to a nucleic acid sequence encoding a constant region of the immunoglobulin heavy chain.

Another exemplary embodiment of a spatial methodology for generating immune cell data is depicted in FIG. 16B. In such embodiments, the capture sequence sequence is a homopolymeric sequence, e.g., a polyT sequence. FIG. 16B shows an exemplary poly(A) capture with a poly(T) capture domain. A poly(T) capture domain can capture other analytes, including analytes encoding ABMs within the tissue sample.

In some embodiments, following capture of analytes by capture probes, capture probes can be extended, e.g., via reverse transcription. Second strand synthesis can generate double stranded cDNA products that are spatially barcoded. The double stranded cDNA products, which may comprise ABM encoding sequences and non-ABM related analytes, can be enriched for ABM encoding sequences.

An exemplary enrichment workflow may comprise amplifying the cDNA products (or amplicons thereof) with a first primer that specifically binds to a functional sequence of the first capture probe or reverse complement thereof and a second primer that binds to a nucleic acid sequence encoding a variable region of the ABM expressed by the ABM-expressing cell or reverse complement thereof. In some embodiments, the first primer and the second primer flank the spatial barcode of the first spatially barcoded polynucleotide or amplicon thereof. In some embodiments, the first primer and the second primer flank a J junction, a D junction, and/or a V junction.

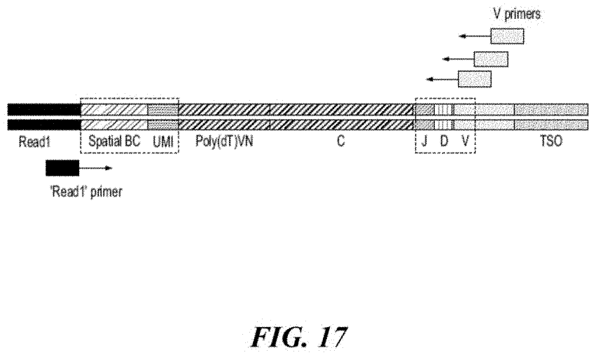

FIG. 17 shows an exemplary analyte enrichment strategy following analyte capture on the array. The portion of the immune cell analyte of interest includes the sequence of the V(D)J region, including CDR sequences. As described herein, a poly(T) capture probe captures an analyte encoding an ABM, an extended capture probe is generated by a reverse transcription reaction, and a second strand is generated. The resulting nucleic acid library can be enriched by the exemplary scheme shown in FIG. 17, where an amplification reaction including a Read 1 primer complementary to the Read 1 sequence of the capture probe and a primer complementary to a portion of the variable region of the immune cell analyte, can enrich the library via PCR. While FIG. 17 depicts a Read 1 primer, it is understood that a primer complementary to other functional sequences, such as other sequencing primer sequences, or sequencer specific flow cell attachment sequences, or portions of such functional sequences, may also be used. While FIG. 17 depicts a polyT capture sequence, it is understood that other capture sequences disclosed herein may be present in library members. The enriched library can be further enriched by nested primers complementary to a portion of the variable region internal (e.g., 5′) to the initial variable region primer for practicing nested PCR.

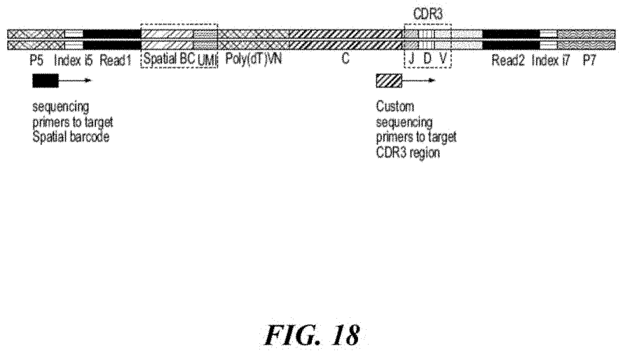

FIG. 18 shows a sequencing strategy with a primer specific complementary to the sequencing flow cell attachment sequence (e.g., P5) and a custom sequencing primer complementary to a portion of the constant region of the analyte. This sequencing strategy targets the constant region to obtain the sequence of the CDR regions, including CDR3, while concurrently or sequentially sequencing the spatial barcode (BC) and/or unique molecular identifier (UMI) of the capture probe. By capturing the sequence of a spatial barcode, UMI and a V(D)J region the receptor is not only determined, but its spatial location and abundance within a cell or tissue is also identified.

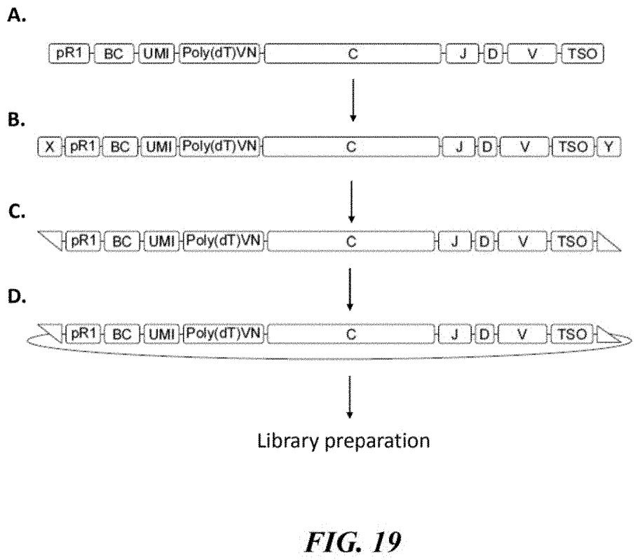

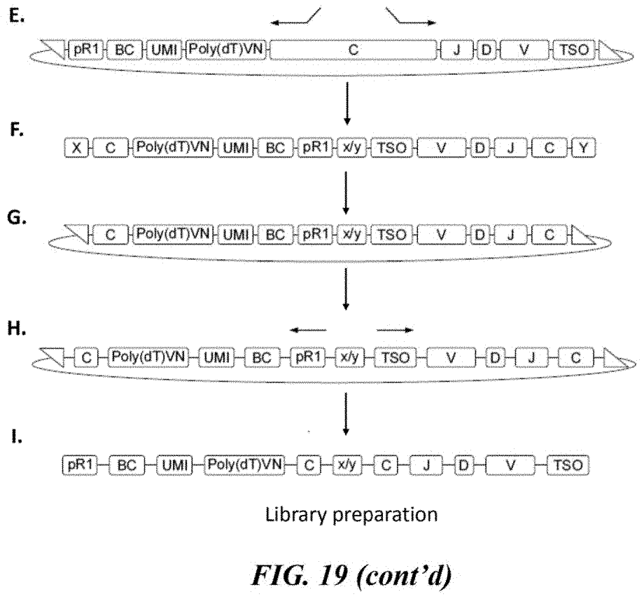

FIG. 19 shows an exemplary nucleic acid library preparation method to remove a portion of an analyte sequence via double circularization of a member of a nucleic acid library. Panel A shows an exemplary member of a nucleic acid library including, in a 5′ to 3′ direction, a first adaptor (e.g., primer sequence R1, pR1 (e.g., Read 1)), a barcode (e.g., a spatial barcode or a cell barcode), a unique molecular identifier (UMI), a capture domain (e.g., poly(T) VN sequence), a sequence complementary to an analyte (C, J, D and V), and a second adaptor (e.g., template switching oligonucleotide sequence (TSO)). For purposes of this example an analyte including a constant region (C) and V(D)J sequence are shown, however, the methods described herein can be equally applied to other analyte sequences in a nucleic acid library. Panel B shows the exemplary member of a nucleic acid library where additional sequences can be added to both the 3′ and 5′ ends of the nucleic acid member (shown as a X and Y) via a PCR reaction. The additional sequences added can include a recognition sequence for a restriction enzyme (e.g., restriction endonuclease). The restriction recognition sequence can be for a rare restriction enzyme. The exemplary member of the nucleic acid library shown in Panel B can be digested with a restriction enzyme to generate sticky ends shown in Panel C (shown as triangles) and can be intramolecularly circularized by ligation to generate the circularized member of the nucleic acid library shown in Panel D. The ligation can be performed with a DNA ligase. The ligase can be T4 ligase. A primer pair can be hybridized to a circularized nucleic acid member, where a first primer hybridizes to a 3′ portion of a sequence encoding the constant region (C) and includes a second restriction enzyme (e.g., restriction endonuclease) sequence that is non-complementary to the analyte sequence, and where a second primer hybridized to a 5′ portion of a sequence encoding the constant region (C), and where the second primer includes a second restriction enzyme sequence (Panel E). The first primer and the second primer can generate a linear amplification product (e.g., a first double-stranded nucleic acid product) as shown in Panel F, which includes the second restriction enzyme recognition sequences (shown as X and Y end sequences). The linear amplification product (Panel F) can be digested with a second restriction enzyme to generate sticky ends and can be intramolecularly ligated with a ligase (e.g., T4 DNA ligase) to generate a second double-stranded circularized nucleic acid product as shown in Panel G. The second double-stranded circularized nucleic product (Panel G) can be amplified with a third primer, pR1, substantially complementary to the first adaptor (e.g., Read 1) sequence and a fourth primer substantially complementary to the second adapter (e.g., TSO) as shown in Panel H to generate a version of the double-stranded member of the nucleic acid library lacking all, or a portion of, the sequence encoding the constant region (C) of the analyte (Panel I). The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, a-tailing, adaptor ligation, and/or additional amplification (e.g., PCR). The fragments can then be sequenced using, for example, paired-end sequencing using TruSeq Read 1 and TruSeq Read 2 as sequencing primer sites or any other sequencing method described herein. As such, sequences can be determined from regions more than about 1 kb away from the end of an analyte (e.g., 3′ end) and can link such a sequence to a barcode sequence (e.g., a spatial barcode, a cell barcode) in library preparation methods (e.g., sequencing preparation). For purposes of this example an analyte including a constant region (C) and V(D)J sequences are shown, however, the methods described herein can be equally applied to other analyte sequences in a nucleic acid library.

An exemplary member of a nucleic acid library can be prepared as shown in FIG. 18 to generate a first double-stranded circularized nucleic acid product shown in Panel D of FIG. 18 as previously described.

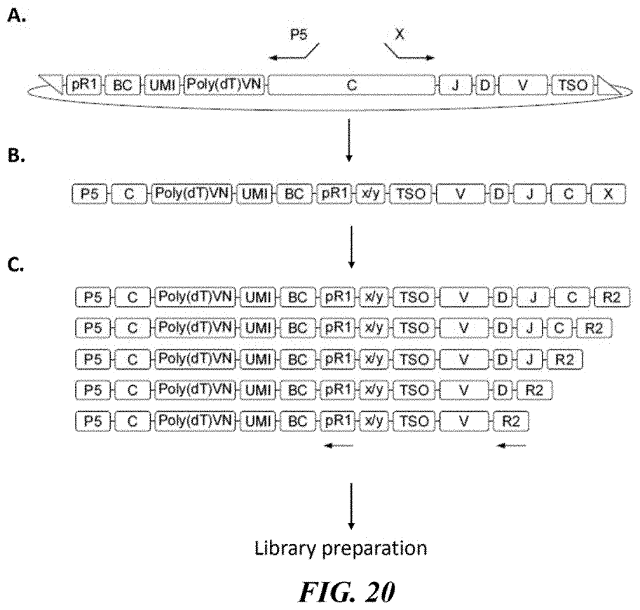

FIG. 20 depicts another exemplary workflow for processing such double-stranded circularized nucleic acid product. A primer pair can be contacted with the double-stranded circularized nucleic acid produce with a first primer that can hybridize to a sequence from a 3′ region of the sequence encoding the constant region of the analyte and a sequence including a first functional domain (e.g., P5). The second primer can hybridize to a sequence from a 5′ region of the sequence encoding the constant region of the analyte, and includes a sequence including a second functional domain (shown as “X”) as shown in Panel A. Amplification of the double-stranded circularized nucleic acid product results in a linear product as shown in Panel B, where all, or a portion of, the constant region (C) is removed. The first functional domain can include a sequencer specific flow cell attachment sequence (e.g., P5). The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites (Panel C, arrows), or any other sequencing method described herein. After library preparation methods described herein, a different sequencing primer for the first adaptor (e.g., Read 1) is used since the orientation of the first adaptor (e.g., Read 1) sequence will be reversed. Accordingly, sequences can be determined from regions more than about 1 kb away from the end of an analyte (e.g., 3′ end) and can link such a sequence to a barcode sequence (e.g., a spatial barcode, a cell barcode) in further library preparation methods (e.g., sequencing preparation). For purposes of this example an analyte including a constant region (C) and V(D)J sequence are shown, however, the methods described herein can be applied to other analyte sequences in a nucleic acid library as well.

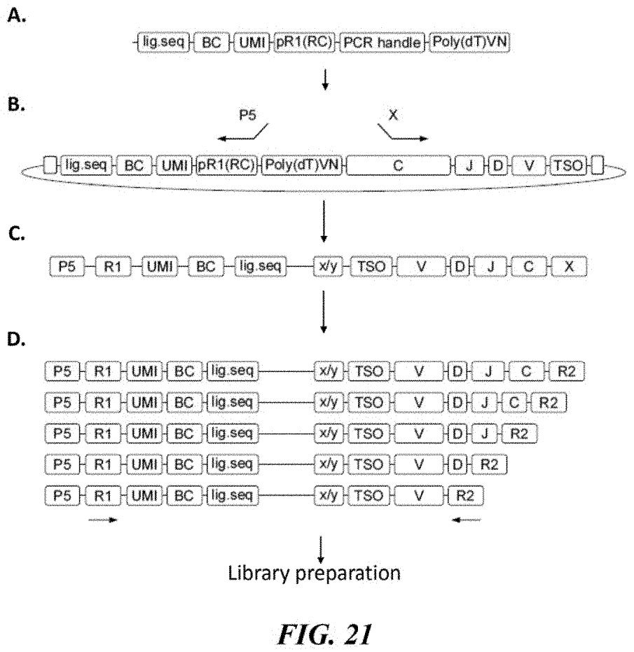

FIG. 21 shows an exemplary nucleic acid library preparation method to remove all or a portion of a constant sequence of an analyte from a member of a nucleic acid library via circularization. Panels A and B shows an exemplary member of a nucleic acid library including, in a 5′ to 3′ direction, a ligation sequence, a barcode sequence, a unique molecular identifier, a reverse complement of a first adaptor (e.g., primer sequence pR1 (e.g., Read 1)), a capture domain, a sequence complementary to the captured analyte sequence, and a second adapter (e.g., TSO sequence). The ends of the double-stranded nucleic acid can be ligated together via a ligation reaction where the ligation sequence splints the ligation to generate a circularized double-stranded nucleic acid as shown in Panel B. The circularized double-stranded nucleic acid can be amplified with a pair of primers to generate a linear nucleic acid product lacking all or a portion of the constant region of the analyte (Panels B and C). The first primer can include a sequence substantially complementary to the reverse complement of the first adaptor and a first functional domain. The first functional domain can be a sequencer specific flow cell attachment sequence (e.g., P5). The second primer can include a sequence substantially complementary to a sequence from a 5′ region of the sequence encoding the constant region of the analyte, and a second functional domain. The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein (Panel D). After library preparation methods (e.g., described herein), sequencing primers can be used since the orientation of Read 1 will be in the proper orientation for sequencing primer pR1. Accordingly, sequences can be determined from regions more than about 1 kb away from the end of an analyte (e.g., 3′ end) and can link such a sequence to a barcode sequence (e.g., a spatial barcode, a cell barcode) in further library preparation methods (e.g., sequencing preparation). For purposes of this example an analyte including a constant region (C) and V(D)J sequence are shown, however, the methods described herein can be applied to other analyte sequences in a nucleic acid library as well.

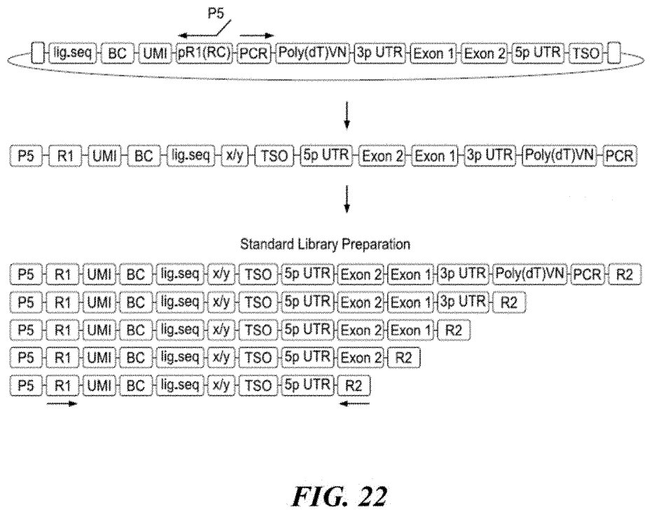

FIG. 22 shows an exemplary nucleic acid library method to reverse the orientation of an analyte sequence in a member of a nucleic acid library. Panel A shows an exemplary member of a nucleic acid library including, in a 5′ to 3′ direction, a ligation sequence, a barcode (e.g., a spatial barcode or a cell barcode), unique molecular identifier, a reverse complement of a first adaptor, an amplification domain, a capture domain, a sequence complementary to an analyte, and a second adapter. The ends of the double-stranded nucleic acid can be ligated together via a ligation reaction where the ligation sequence splints the ligation to generate a circularized double-stranded nucleic acid also shown in Panel A. The circularized double-stranded nucleic acid can be amplified to generate a linearized double-stranded nucleic acid product, where the orientation of the analyte is reversed such that the 5′ sequence (e.g., 5′ UTR) is brought in closer proximity to the barcode (e.g., a spatial barcode or a cell barcode) (Panel B). The first primer includes a sequence substantially complementary to the reverse complement of the first adaptor and a functional domain. The functional domain can be a sequencer specific flow cell attachment sequence (e.g., P5). The second primer includes a sequence substantially complementary to the amplification domain. The resulting double-stranded member of the nucleic acid library including a reversed analyte sequence (e.g., the 5′ end of the analyte sequence is brought in closer proximity to the barcode) can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein. Accordingly, sequences from the 5′ end of an analyte will be included in sequencing libraries (e.g., paired end sequencing libraries). Any type of analyte sequence in a nucleic acid library can be prepared by the methods described in this Example (e.g., reversed).

Data Analysis

Data analysis 140 includes processing and analyzing sequence dataset 132. This analysis may be performed in any number of different ways to extract various pieces of information from sequence dataset 132. Various methods and systems may be employed to analyze sequence dataset 132 received as input in accordance with one or more embodiments described herein. Data analysis 140 may generate any number of outputs that may be referred to as a dataset, sequencing output data, or sequencing information.

In one or more embodiments, data analysis 140 may be implemented using hardware, software, firmware, or a combination thereof. For example, data analysis 140 may be implemented using computing platform 142. Computing platform 142 may include a computer system, a cloud computing platform, one or more processing units, some other type of computing platform, or a combination thereof. The computer system may include a single computer or multiple computers in communication with each other. In one or more embodiments, one or more functions performed by of computing platform 142 may be implemented using one or more computing nodes in a cloud processing service (e.g., AMAZON WEB SERVICES™).

In one or more embodiments, computing platform 142 is communicatively coupled (e.g., via direct wired connection(s) or wireless connection(s)) to data store 134, display system 144, set of input devices 146, or a combination thereof. In one or more embodiments, display system 144, one or more input devices of set of input devices 146, or both are at least partially integrated within computing platform 142.

In other embodiments, display system 144, one or more input devices of set of input devices 146, or a combination thereof may be separate from but in communication with computing platform 142. Computing platform 142 may receive, retrieve, or otherwise obtain sequence dataset 132 from data store 134. Display system 144 may be used to, for example, without limitation, visualize sequence dataset 132, visualize information generated via data analysis 140, or both. Set of input devices 146 enable a user to provide user input for utilization during data analysis 140.

Any combination or configuration of computing platform 142, data store 134, display system 144, or set of input devices 146 may be integrated into a system assembly (e.g., housed in a same housing and/or communicatively coupled via conventional device/component connection means). For example, in various embodiments, computing platform 142, data store 134, display system 144, one or more input devices of set of input devices, or a combination thereof may be housed in a same housing assembly and communicate via one or more communications links (e.g., wired communications links, wireless communications links, optical communications links, etc.). In various embodiments, computing platform 142 may be connected via a LAN or WAN connection that allows for the transmission of data to and from data store 134, one or more input devices of set of input devices 146, display system 144, or a combination thereof.

In one or more embodiments, data analysis 140 includes processing sequencing dataset 132 using. Cell Ranger (software that is available from 10× Genomics). Cell Ranger may, for example, process sequencing dataset 132 and transform sequencing dataset 132 into one or more datasets that are ready for analysis by the various embodiments, systems and methods within the disclosure. Cell Ranger is one example of an implementation for software that may be used to process and/or analyze sequencing dataset 132.

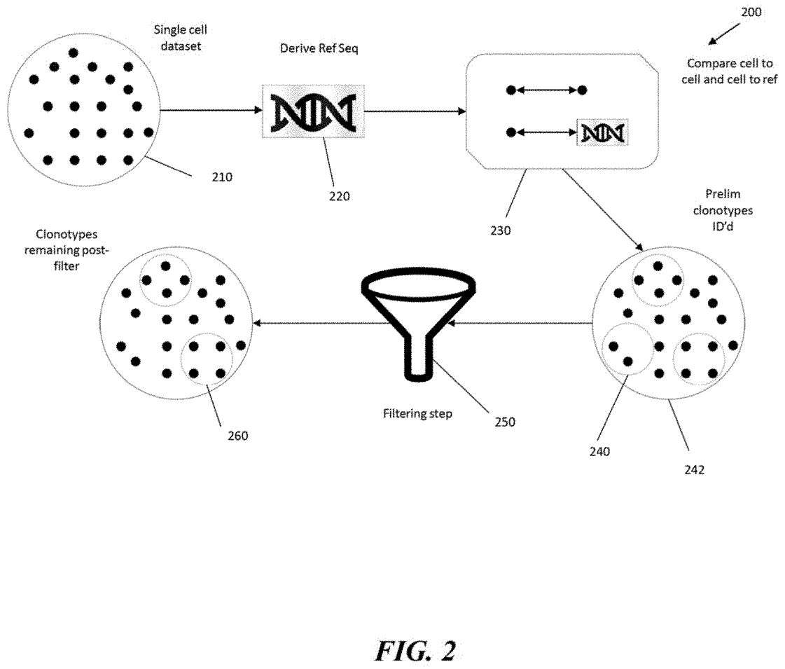

In some cases, data analysis 140 with respect to the various embodiments, systems, and methods described herein may include processing sequence dataset 132 to identify clonotypes and group cells into clonotype groups. A clonotype group is thus a group of cells that belong to a same clonotype. One example of a manner in which clonotyping may be performed is described below in FIG. 2. In various embodiments, clonotyping may be performed using a software tool or module. This software tool or module may include, for example, the enclone software tool (available from 10× Genomics). In some embodiments, at least a portion of the enclone software tool is integrated with Cell Ranger. In other embodiments, the enclone software tool is integrated within some other type of software or implemented independently and separate from other software tools.

### IV. Clonotyping

FIG. 2 is a schematic diagram of a workflow 200 for clonotyping in accordance with one or more embodiments. Workflow 200, which may also be referred to as a clonotyping workflow, is one example of a manner in which clonotyping may be performed as part of data analysis 140 from FIG. 1. In one or more embodiments, workflow 200 is implemented within computing platform 142 in FIG. 1. Workflow 200 may be used to group immune cells identified within an immune cell sequence dataset 202 into one or more clonotypes. In some embodiments, workflow 200 may include other features or steps in addition to or in place of the ones described in FIG. 2. In some embodiments, workflow 200 may include fewer features or steps than those shown in FIG. 2.

Workflow 200 includes processing an immune receptor dataset (e.g, an immune cell sequence dataset) 210, which may also be referred to as a lymphoid cell sequence dataset. In some embodiments, the immune receptor dataset 210 can comprise VDJ sequence information. In some embodiments, the immune receptor dataset 210 can be a variable domain region sequence data set, e.g., obtained from a cell sample comprising VDJ expressing cells, e.g., a cell sample comprising a plurality of lymphoid cells. Immune cell sequence dataset 210 may be, may be part of, or may be derived from sequencing dataset 132 described above in FIG. 1. As previously described, an immune cell may be any cell of the immune system such as, for example, without limitation, a B cell, a T cell, natural killer (NK) cell, or some other type of cell of the immune system. In one or more embodiments, immune cell sequence dataset 210 includes an immune cell receptor sequence dataset. The immune cell receptor sequence dataset includes a plurality of immune cell receptor sequences, at least one of which (or each of which) may be associated with a given immune cell. Multiple immune cell receptor sequences may be associated with a same immune cell. In one or more embodiments, the immune cell receptor sequence dataset includes one or more immune cell receptor variable region sequences, one or more immune and/or constant region sequence(s).

From immune cell sequence dataset 210, reference immune cell receptor sequence(s) 220 may be derived. Reference immune cell receptor sequence(s) 220 may include a donor reference sequence, a universal reference sequence, or both. With immune cell sequence dataset 210 and immune cell receptor sequence(s) 220 in hand, one or more comparisons 230 may be conducted. These one or more comparisons 230 may include comparing the different immune cell receptor sequences (e.g., TCR variable region sequences, BCR variable region sequences, etc.) associated with the immune cells of immune cell sequence dataset 210. In one or more embodiments, the one or more comparisons 230 may include comparing the variable region sequences of the immune cells to the reference immune cell receptor sequence(s) 220. Again, various reference to cell comparisons can be contemplated here and will be discussed in further detail below. It should be understood, and will be discussed below, that both comparisons are individually beneficial for grouping purposes, but can also be done together as part of the workflow.

Based on the one or more comparisons 230, one or more clonotypes 240 can be identified from immune cell sequence dataset 210, as part of an identification protocol 242. Via identification protocol 242, the identification of clonotypes 240 is subject to meeting one or more comparison criteria. Detail regarding how comparisons 230, via the one or more comparison criteria, can lead to identification of the one or more clonotypes 240, will be provided below.

Identified clonotypes 240 can also be subject to one or more filters 250 that can function to remove specific cells from identified clonotypes, or eliminate whole clonotypes, that do not meet specific comparison criteria or are filtered out via the constraints imposed by the one or more filters 250. Detail regarding the filters will be provided below. Again, it should be understood that FIG. 2 simply illustrates a non-limiting example of the process for grouping lymphoid cells. As such, the one or more filters 250 can activate after clonotypes are identified. Alternatively, the one or more filters can activate as part of identification protocol 242. Moreover, it is contemplated that one or more of filters 250 can activate before identification protocol 242. Even further, there need not be any active filters as part of the workflow 200.

Regardless of when or if one or more filters 250 are activated, an updated set of clonotypes 260 can be identified. As illustrated in FIG. 2, after application of filter(s) 250, two clonotypes 260 remained of the three originally identified clonotypes 240. It is understood, however, that in accordance with various embodiments, the one of more filters 250 need not be used, and that identification of the updated set of clonotypes 260 need not occur.

### V. Visualization of Antigen Binding Profiles of Cells in Clonotype(s)

As previously described, the various embodiments described herein provide methods and systems for providing a visualization of the antigen binding profiles of immune cells. In one or more embodiments, this visualization is provided with respect to a clonotype. In some embodiments, this visualization is provided for multiple clonotypes. In various embodiments, the visualization is generated in the form of one or more binding diagrams that can be displayed to a user to enable the user to readily and easily discern important information about the multi-antigen binding capabilities of a given clonotype, a group of clonotypes, or both. In some cases, the one or more binding diagrams enable a user to make conclusions about cross-reactivity among the immune cells.

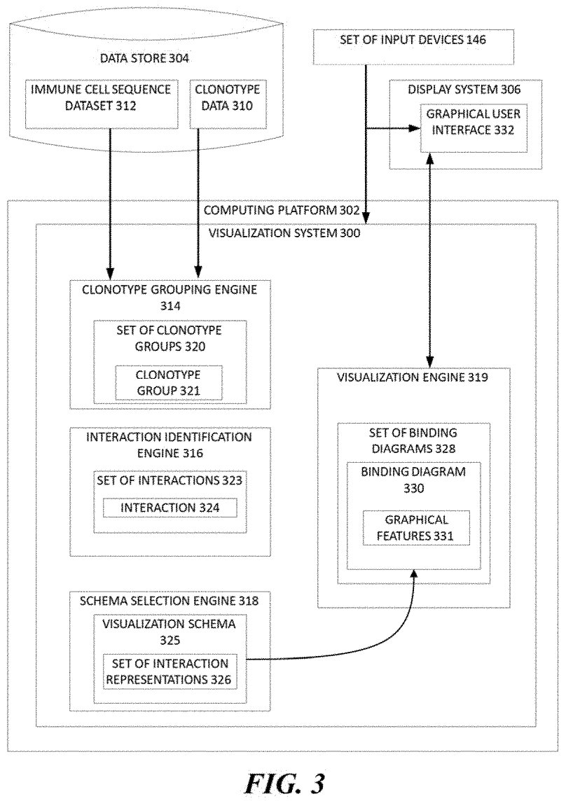

FIG. 3 is a schematic diagram of a visualization system in accordance with one or more embodiments. Visualization system 300 is used to visualize the antigen binding profiles of immune cells. Visualization system 300 may be implemented using hardware, software, firmware, or a combination thereof. In one or more embodiments, visualization system 300 is implemented using computing platform 302. In one or more embodiments, computing platform 302 takes the form of computing platform 142 in FIG. 1. In other embodiments, computing platform 302 takes the form of a computing platform implemented in a manner similar to computing platform 142 in FIG. 1. For example, computing platform 142 may include one or more processing units, a computer system, a cloud computing device, another type of computing device, or a combination thereof.

Visualization system 300 may be communicatively coupled (e.g., via one or more wired connections, wireless connections, optical connections, etc.) to data store 304, display system 306, set of input devices 308, or a combination thereof. In one or more embodiments, one or more of data store 304, display system 306, and set of input devices 308 may be implemented in the form of data store 134, display system 144, and set of input devices 146, respectively, in FIG. 1. In other embodiments, data store 304, display system 306, set of input devices 308, or a combination thereof can be integrated within or as part of visualization system 300.

Data store 304 may store clonotype data 310. In one or more embodiments, clonotype data 310 includes information about one or more clonotype groups identified from immune cell sequence dataset 312. In some embodiments, immune cell sequence dataset 312 is stored in data store 304. In other embodiments, immune cell sequence dataset 312 is stored in another data store separate from data store 304. Immune cell sequence dataset 312 may be one example of an implementation for immune cell sequence dataset 210 in FIG. 2 or one example of at least a portion of data in, generated from, or otherwise derived from sequence dataset(s) 132. Clonotype data 310 may be one example of an implementation for data that identifies clonotypes 240, updated set of clonotypes 260, or both from FIG. 2.

Visualization system 300 may include, for example, without limitation, clonotype grouping engine 314, interaction identification engine 316, schema selection engine 318, visualization engine 319, or a combination thereof. At least one (or each) of these different engines may be implemented using hardware, firmware, software, or a combination thereof. These engines may also be referred to as modules in some cases. Although each of clonotype grouping engine 314, interaction identification engine 316, schema selection engine 318, and visualization engine 319 is described independently below, in one or more embodiments, at least a portion of the functions of multiple ones of these engines may be integrated together. For example, in some embodiments, the functions of schema selection engine 318 and visualization engine 319 may be integrated together as a single engine or module within visualization system 300. In other embodiments, the functions of clonotype grouping engine 314 and interaction identification engine 316 may be integrated together.

In one or more embodiments, clonotype grouping engine 314 is used to group immune cells identified in immune cell sequence dataset 312 into set of clonotype groups 320. For example, clonotype grouping engine 314 may obtain (e.g., receive, retrieve, etc.) immune cell sequence dataset 312 from data store 304 and may identify set of clonotype groups 320 based on immune cell sequence dataset 312. Set of clonotype groups 320 includes one or more clonotype groups. A clonotype group includes a group of individual immune cells that have been identified as belonging to a same clonotype. In some embodiments, clonotype grouping engine 314 generates clonotype data 310 that identifies and includes information about set of clonotype groups 320. Clonotype grouping engine 314 can store clonotype data 310 in data store 304 for use by visualization system 300 or some other system at a later point in time.

In other embodiments, clonotype grouping engine 314 is used to obtain clonotype data 310 that identifies set of clonotype groups 320 from data store 304. For example, clonotype data 310 may have been generated by another system or engine outside of visualization system 300. In these cases, clonotype grouping engine 314 may retrieve clonotype data 310 from data store 304. In some embodiments, clonotype data 310 is in a format that can be processed by interaction identification engine 316. In one or more embodiments, clonotype data 310 is preprocessed by clonotype grouping engine 314 into a format improved use by interaction identification engine 316.

Interaction identification engine 316 may process at least a portion of clonotype data 310 to identify at least one interaction between at least one cell of a clonotype group in set of clonotype groups 320 and multiple antigens. For example, clonotype data 310 may include antigen binding information that identifies the one or more antigens to which a cell (or each cell) of the clonotype group binds or is believed to bind.

Clonotype group 321 is one example of a clonotype group in set of clonotype groups 320. For clonotype group 321, interaction identification engine 316 may identify set of interactions 323 associated with one or more cells of clonotype group 321. Interaction 324 is one example of an interaction in set of interactions 323. In one or more embodiments, interaction 324 includes the binding of a cell (or each cell of these one or more cells) with at least one (or each) of a plurality of antigens (or a particular combination of antigens). In this manner, interaction 324 may be a multi-antigen binding interaction. This plurality of antigens may be a subset of the various antigens associated with the particular sample (e.g., sample 112 in FIG. 1) for which clonotype data 310 was generated. In various embodiments, interaction 324 may be referred to as a multi-antigen binding interaction that is likely to occur with at least one (or each) of the subset of cells based on the antigen binding information included in clonotype data 310. Further, interaction identification engine 316 may be used to identify at least one interaction between at least one cell of the set of individual immune cells in clonotype group 321 and at least one antigen of a plurality of antigens.

Schema selection engine 318 is used to select visualization schema 325 to visualize the set of interactions 123 identified by interaction identification engine 316. In one or more embodiments, visualization schema 325 includes a different interaction representation for individual interactions of the set of interactions 323 (or for each interaction of the set of interactions 323) to enable the various interactions to be visually distinguished from each other. For example, visualization schema 325 selected by schema selection engine 318 may include interaction representation 326 to visualize represent interaction 324. Interaction representation 326 may include one or more graphical features (e.g., graphical representations, graphical indicators, graphical components, etc., or combination thereof) that are selected to visually provide information about interaction 324.

Visualization engine 319 is used to create a set of visualization that graphically represents the multi-antigen binding of the various cells in set of clonotype groups 320. In one or more embodiments, this set of visualizations is implemented in the form of set of binding diagrams 328. In various embodiments, a binding diagram can be created for a clonotype group (or for each clonotype group) in the set of clonotype groups 320. In other embodiments, a binding diagram is created for a subset of cells in a clonotype group. In still other embodiments, a binding diagram is created for the combination of cells belonging to two or more clonotype groups 320. In this manner, set of binding diagrams 328 may be created in association with set of clonotype groups 320 in various ways.

Binding diagram 330 is one example of a binding diagram in set of binding diagrams 328. Binding diagram 330 may be created for, for example, clonotype group 321. Binding diagram 330 is generated with visualization schema 325 such that binding diagram 330 can be used to visually provide information about set of interactions 323. Set of binding diagrams 328 provide a visualization of the various antigen binding profiles for set of clonotype groups 320. For example, binding diagram 330 provides the antigen binding profile (with respect to multi-antigen binding capabilities) for clonotype group 321.

Binding diagram 330 may include, for example, graphical features 331. Graphical features 331 may include any number or combination of graphical representations, graphical indicators, graphical components, or other types of graphical or visual elements that aid in visualization. Graphical features 331 may include at least set of interaction representations 326. In other embodiments, graphical features 331 include indicators corresponding to antigens in which a particular indicator provides a visual indication of the number of cells in clonotype group 321 capable of binding to the corresponding antigen, the proportion of cells in clonotype group 321 that bind to the corresponding antigen, or both.

In one or more embodiments, visualization engine 319 may render (or display) binding diagram 330 in graphical user interface 332 on display system 306. Binding diagram 330 may provide a user with a visual understanding of the multi-antigen binding of cells within clonotype group 321. Further, binding diagram 330 may be created such that the user may readily and easily reach this visual understanding, while being able to easily and ready visually distinguish between different interactions of set of interactions 323. In other embodiments, visualization engine 319 may render the entire set of binding diagrams 328 in graphical user interface 332 simultaneously to provide a user with an overall picture of the multi-antigen binding of cells across set of clonotype groups 320.

In still other embodiments, visualization engine 319 may render set of binding diagrams 328 for display in graphical user interface 332 in a manner that enables the user to view, and in some cases, interact with, a binding diagram (or each binding diagram) in the set of binding diagrams 328 independently. The user may use, for example, one or more input devices of set of input devices 308 to interact with graphical user interface 332 and the one or more binding diagrams displayed in graphical user interface 332. In some embodiments, at least a portion of the functions performed by visualization engine 319 may be implemented within display system 306.

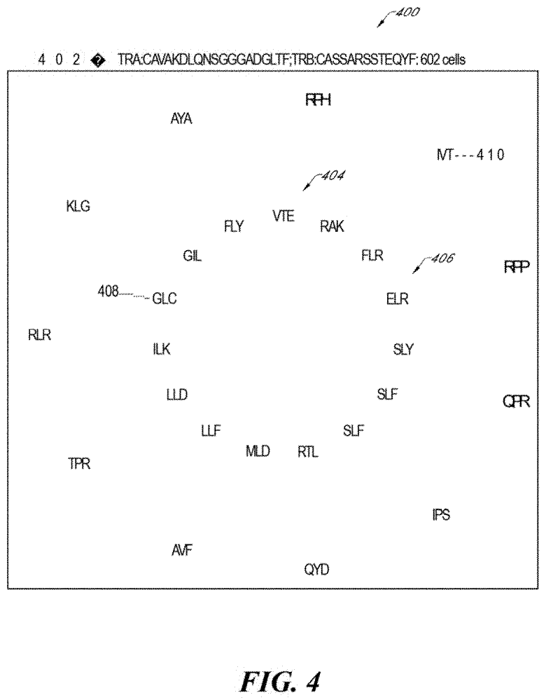

FIGS. 4-6 illustrate the various stages in the generation of a binding diagram in accordance with one or more embodiments. FIG. 4 is an illustration of a binding diagram 400 in accordance with one or more embodiments. Binding diagram 400 may be one example of an implementation for binding diagram 330 in FIG. 3 generated by visualization system 300 in FIG. 3.

Binding diagram 400 includes clonotype group information 402 and collection of antigens 404. Clonotype group information 402 identifies, for example, without limitation, the clonotype group (e.g., clonotype group 321 in FIG. 3) with which binding diagram 400 is associated. In one or more embodiments, clonotype group information 402 also identifies the number of cells (e.g., immune cells such as T cells) that belong to that clonotype group.

Collection of antigens 404 are presented with spatial arrangement 406 that provides information about the antigens within collection of antigens 404. Spatial arrangement 406 may be one example of a portion of visualization schema 325 described with respect to FIG. 3. Collection of antigens 404 may include antigens of interest such as, for example, but not limited to, those antigens identified as binding to the cells of one or more clonotype groups of interest. Spatial arrangement 406 divides collection of antigens 404 into a first portion, first group of antigens 408, and a second portion, second group of antigens 410. In one or more embodiments, spatial arrangement 406 includes a concentric circle-type arrangement in which first group of antigens 408 are displayed in the form of an inner circle and second group of antigens 410 are displayed in the form of an outer circle around first group of antigens 408.

First group of antigens 408 includes those antigens for which HLA alleles associated with first group of antigens 408 match HLA alleles expressed by the immune cells in the clonotype group. Second group of antigens 410 includes those antigens for which HLA alleles associated with second group of antigens 410 do not match the HLA alleles expressed by the immune cells in the clonotype group. Spatial arrangement 406 enables a user to readily and easily visually distinguish between first group of antigens 408 and second group of antigens 410.

Although binding diagram 400 is shown in FIG. 4 with respect to a clonotype of T cells, binding diagram 400 could similarly be used for a clonotype of B cells. For example, with B cells, spatial arrangement 406 may be used to visualize some other characteristic of collection of antigens 404. For example, spatial arrangement 406 may be used to visualize the source or source category for the various antigens in collection of antigens 404.

As one example, first group of antigens 408 may include various spike protein mutants from a SARS-associated coronavirus (e.g., from SARS-CoV-2, from SARS-CoV-1, from both, etc.); second group of antigens 410 may include various spike protein mutants from non-SARS coronaviruses. In this example, spatial arrangement 406 may position first group of antigens 408 in the inner circle and second group of antigens 410 in the outer circle. Of course, in other embodiments, some other type of spatial arrangement 406 that enables the source categories (e.g., SARS-associated and non-SARS coronaviruses) to be visually distinguished may be used.

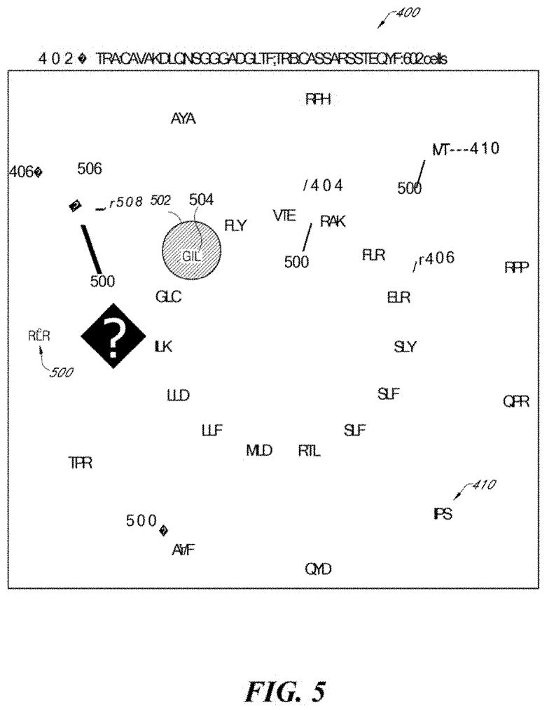

FIG. 5 is an illustration of binding diagram 400 with additional information in accordance with one or more embodiments. Set of graphical features 500 have been added to binding diagram 400. Set of graphical features 500 may be one example of an implementation for at least a portion of the graphical features 331 described with respect to FIG. 3. Further, set of graphical features 500 may be one example of a portion of visualization schema 325 described with respect to FIG. 3.

At least one graphical feature (or each graphical feature) of set of graphical features 500 is a representation or indicator that corresponds to an antigen in collection of antigens 404. For example, set of graphical features 500 is used to provide information about the corresponding set of antigens. In one or more embodiments, at least one graphical feature (or each graphical feature) of set of graphical features 500 provides a visual indication of at least one of the number of cells in the clonotype group that bind to the corresponding antigen or the proportion of cells in the clonotype group that bind to the corresponding antigen.

Graphical indicator 502 is one example of a graphical feature in set of graphical features 500. Graphical indicator 502 is displayed in relation to its corresponding antigen, antigen 504. Antigen 504 belongs to first group of antigens 408. Graphical indicator 502 may be, for example, but is not limited to, a shape indicator (e.g., a circle, a triangle, a rectangle, a square, an oval, a star, or some other type of specific or nonspecific shape).

In one or more embodiments, graphical indicator 502 takes the form of a circle that has a size that visually indicates the number of cells in the clonotype group that bind to the corresponding antigen. For example, the larger the size of graphical indicator 502, the greater the number of cells that bind to antigen 504. In one or more embodiments, graphical indicator 502 has a pattern, texture, shading, color, or other graphical feature that visually indicates the proportion of cells in the clonotype group that bind to antigen 504. As one example, a darker color or shade or a denser pattern for graphical indicator 502 may indicate a higher proportion of cells in the clonotype group that bind to antigen 504. In some embodiments, a lack of a color, shade, pattern, texture, or other graphical feature for a graphical indicator, such as with graphical indicator 506 associated with antigen 508, may indicate that the proportion of cells in the clonotype group that bind to that antigen is below some threshold proportion (e.g., below 10%, below 15%, below 20%, below 5%, etc.).

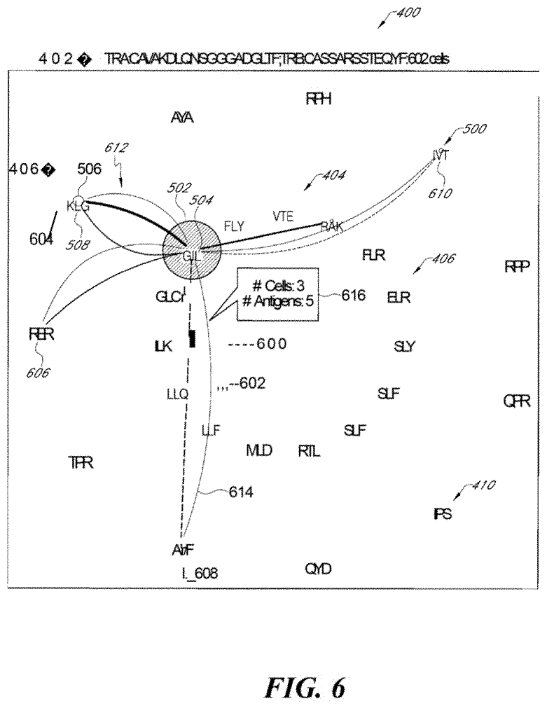

FIG. 6 is an illustration of binding diagram 400 with additional information in accordance with one or more embodiments. In FIG. 6, set of interaction representations 600 have been added to binding diagram 400. Set of interaction representations 600 may be one example of an implementation for set of interaction representations 326 and thereby, for at least a portion of visualization schema 325 and graphical features 331 described with respect to FIG. 3. Set of interaction representations 600 is used to visually represent a set of interactions in which an interaction (or each interaction) is between one or more immune cells and multiple antigens. In particular, one of the one or more immune cells binds to one of the multiple antigens to form the “interaction.” Alternatively, each of the one or more immune cells binds to each of the multiple antigens to form the “interaction.”

For example, set of interaction representations 600 includes interaction representation 602. Interaction representation 602 represents the interaction between a set of cells and plurality of antigens 604. Plurality of antigens 604 includes antigen 504, antigen 508, antigen 606, antigen 608, and antigen 610.

In one or more embodiments, interaction representation 602 includes set of graphical features (e.g., lines, edges, curves, connectors, etc.) 612 that relate the different antigens of plurality of antigens 604 together. In one or more embodiments, set of graphical features 612 is further selected to visually indicate the number of cells in the set of cells that all bind to the same plurality of antigens 604. For example, when set of graphical features 612 takes the form of a set of edges, the thickness of one or more of the edges (or each edge) may indicate the number of cells in the set of cells that all bind to the same plurality of antigens 604.

In various embodiments, at least one (or each) interaction representation of set of interaction representations 600 is visually distinguishable from at least one other interaction representation, or, in various embodiments, visually distinguishable from each other. For example, in some cases, an interaction representation (or each interaction representation) of set of interaction representations 600 may have a distinct pattern, texture, shade, pattern, color, or other graphical feature that allows it to be distinguished from another interaction representation of set of interaction representations 600.

In some embodiments, binding diagram 400 is interactive. For example, a user may be able to interact with binding diagram 400 in a manner that causes additional information to be displayed. As one example, a user may use an input device to hover a cursor over graphical feature 614 (e.g., a line, a curve, an edge, a connector, etc.) that represents a particular interaction, thereby causing graphical element 616 to be displayed. Graphical element 616 may, for example, provide details about the subset of cells associated that particular interaction. For example, graphical element 616 may be a text box or window that identifies the number of cells associated with that particular interaction. In some cases, graphical element 616 may identify the number of antigens associated with that particular interaction.

In this manner, binding diagram 400 is generated in a manner that is able to visually present a sizeable amount of information about the cells in the clonotype group in a condensed and efficient visual manner that enables a user to readily and easily discern multi-antigen binding specificities for the various cells in the clonotype group. Further, in various embodiments, binding diagram 400 is presented in a manner that reduces the overall computing resources and visual space (e.g., space within a graphical user interface such as graphical user interface 332 in FIG. 3) that might otherwise be needed to present the same information in a different form (e.g., a listing, a table, a spreadsheet, etc. identifying the multi-antigen binding for a cell, or each cell, in the clonotype group).

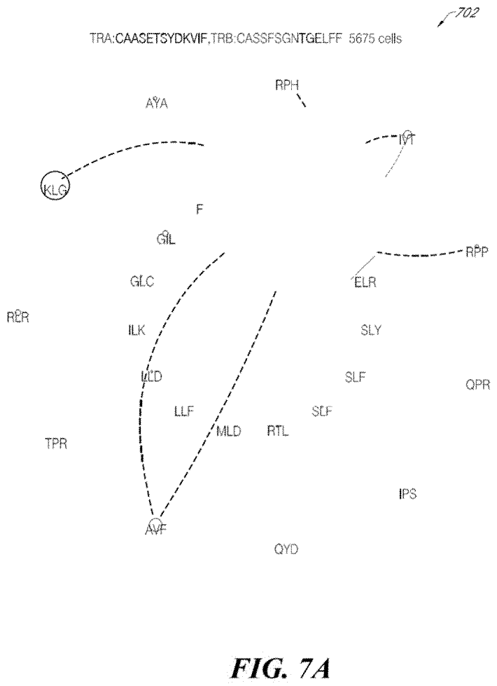

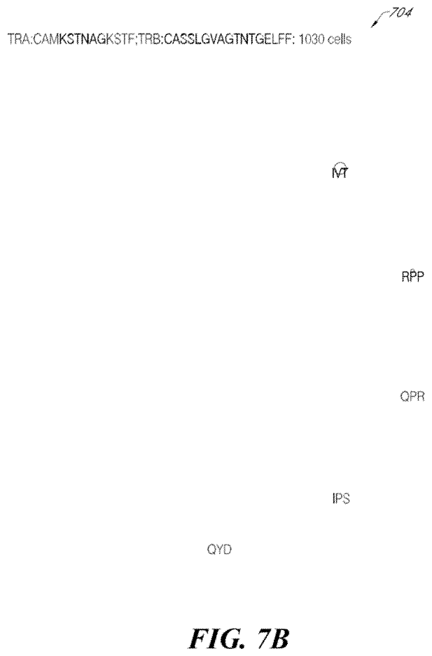

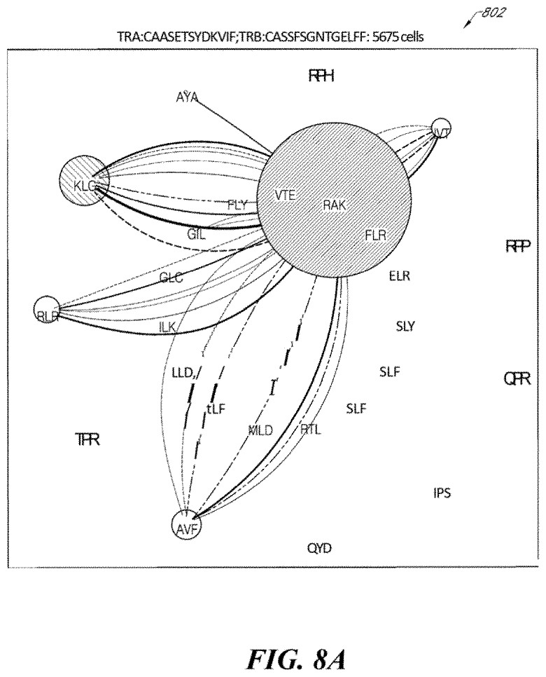

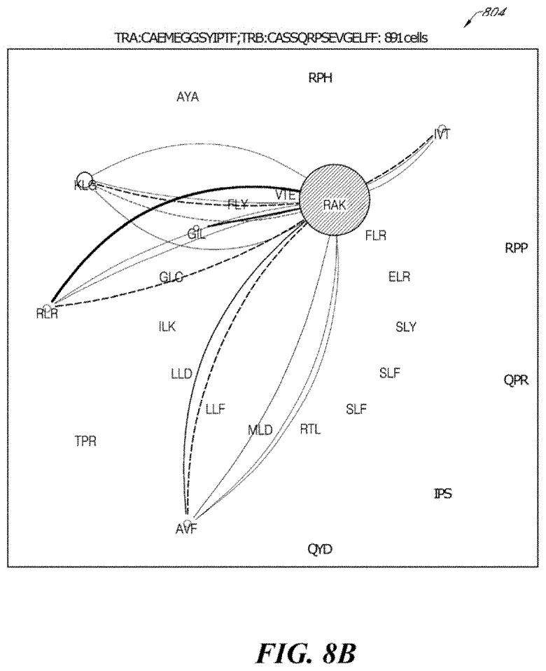

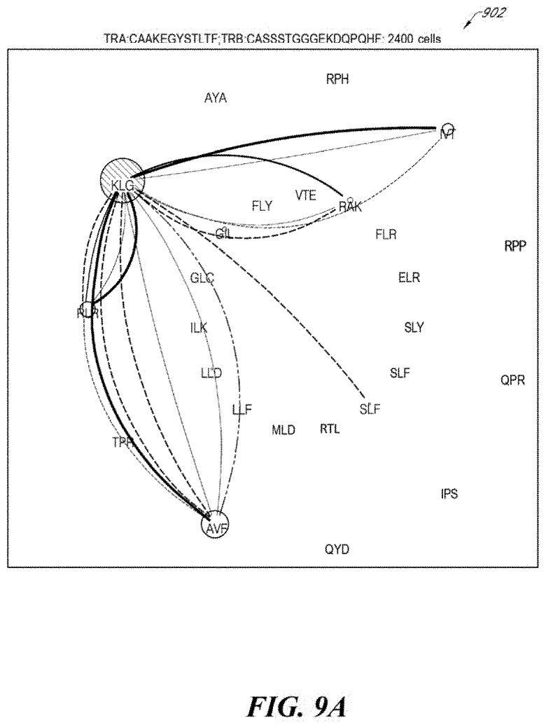

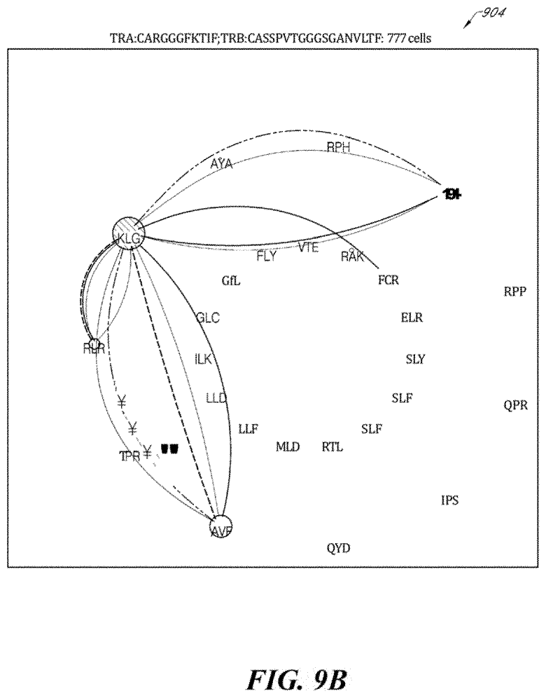

FIGS. 7-9 are illustrations of other binding diagrams in accordance with one or more embodiments. FIGS. 7A and 7B include binding diagram 702 and binding diagram 704. FIGS. 8A and 8B include binding diagram 802 and binding diagram 804. FIG. 9 includes binding diagram 902 and binding diagram 904. Binding diagram 400 in FIG. 6, binding diagrams 702 and 704 in FIG. 7, binding diagrams 802 and 804 in FIG. 8, and binding diagrams 902 and 904 in FIGS. 9A and 9B together form one example of an implementation for at least a portion of the binding diagrams in set of binding diagrams 328 in FIG. 3. These different binding diagrams, which corresponds to a different clonotype group, are visually distinct and these visual differences can enable a user to readily and easily understand the antigen-binding characteristics of the cells in that clonotype group.

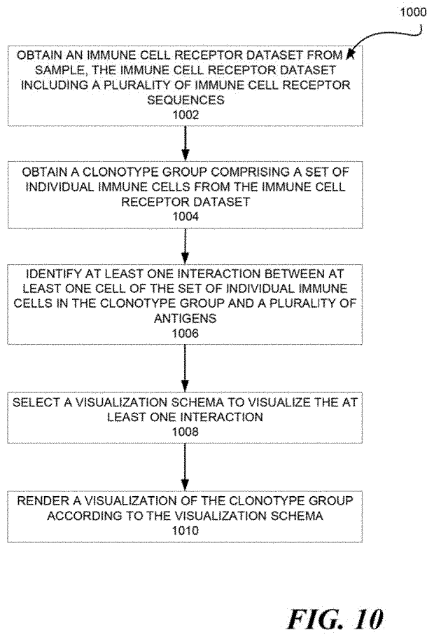

FIG. 10 is a flow chart of a method 1000 for visualizing immune cells within an immune cell receptor dataset in accordance with various embodiments. Method 1000 may be implemented using, for example, without limitation, visualization system 300 in FIG. 3.

Step 1002 may include obtaining an immune cell receptor dataset from a sample, the immune cell receptor dataset including a plurality of immune cell receptor sequences. In one or more embodiments, the immune cell receptor dataset in step 1002 is included in immune cell sequence dataset 210 from FIG. 2, immune cell sequence dataset 312 from FIG. 3, or another immune cell sequence dataset. In step 1002, an immune cell receptor sequence (or, in some cases, each sequence) is associated with an individual immune cell in the sample. In one or more embodiments, the sample is a biological sample of tissue. For example, the biological sample may be extracted from a diseased subject, a vaccinated subject, a healthy subject, an immunosuppressed subject, a subject having an autoimmune disorder or issue. In some cases, the biological sample may be extracted from a subject at a timepoint prior to a disease outbreak. In other cases, the biological sample may be extracted from a population during a disease outbreak, regardless of whether the subject or population is symptomatic or asymptomatic.

In one or more embodiments, at least one immune cell receptor sequence of the plurality of immune cell receptor sequences is a sequence for a T cell receptor associated with a T cell or a B cell receptor associated with a B cell. In various embodiments, at least one immune cell receptor sequence of the plurality of immune cell receptor sequences includes at least one heavy chain region sequence, at least one light chain region sequence, or a combination thereof. In various embodiments, the immune cell receptor dataset obtained in step 1002 includes antigen binding information for the plurality of immune cell receptor sequences.

Step 1004 may include obtaining a clonotype group comprising a set of individual immune cells from the immune cell receptor dataset. In various embodiments, step 1004 may include comparing the immune cell receptor sequences associated with a first immune cell and with a second immune cell from the sample. Further, step 1004 may include identifying the first immune cell and the second immune cell as members of the same clonotype or clonotype group if a set of immune cell receptor sequence comparison criteria is met. In various embodiments, step 1004 may be performed by clonotype grouping engine 314 in FIG. 3.

Step 1006 may include identifying at least one interaction between at least one cell of the set of individual immune cells in the clonotype group and a plurality of antigens. In various embodiments, step 1006 may be performed by interaction identification engine 316 in FIG. 3. In one or more embodiments, the at least one interaction includes a plurality of interactions between at least one cell of the set of individual immune cells in the clonotype group and a plurality of antigens. In various embodiments, the at least one interaction comprises a plurality of interactions between a plurality of cells of the set of individual immune cells in the clonotype group and a plurality of antigens. Set of interactions 323 in FIG. 3 may be one example of the at least one interaction that can be identified by step 1006.

In one or more embodiments, step 1006 includes using the antigen binding information in the immune cell receptor dataset to identify the at least one interaction. In various embodiments, step 1006 includes determining at least one cell as binding to an antigen if the at least one cell has at least a pre-determined number (e.g., a threshold number) of copies of immune cell receptor sequences that bind the antigen. For example, without limitation, a cell may be determined as binding to an antigen if the cell has at least ten immune cell receptor sequences that have been identified as binding the antigen. In other embodiments, some other threshold number (e.g., 5, 8, 12, 14, 15, etc.) may be selected.

Step 1008 may include selecting a visualization schema to visualize the at least one interaction. In various embodiments, step 1008 may be performed by schema selection engine 318 in FIG. 3. Visualization schema 325 in FIG. 3 is one example of an implementation for the visualization schema selected in step 1008. In one or more embodiments, step 1008 includes selecting a spatial arrangement of the plurality of antigens based on the relationship between first human leukocyte antigen (HLA) alleles present in the plurality of antigens and second HLA alleles expressed on the immune cells. In one or more embodiments, this spatial arrangement includes selecting a spatial arrangement that enables visually distinguishing from a first portion of the plurality of antigens in which the first HLA alleles associated with the first portion match the second HLA alleles expressed by the immune cells and a second portion of the plurality of antigens in which the first HLA alleles associated with the second portion do not match the second HLA alleles expressed by the immune cells.

In various embodiments, step 1008 includes selecting a representation for at least a portion of the plurality of antigens. The representation for a particular antigen may, for example, represent information that includes at least one of a number of cells in the clonotype group that bind to the particular antigen, a proportion of cells in the clonotype group that bind to particular antigen, or some other type of information. For example, without limitation, the representation for the particular antigen may be a graphical representation (e.g., using size, color, shading, pattern, etc.) selected to visually indicate at least one of the number of cells in the clonotype group that bind to the particular antigen, a proportion of cells in the clonotype group that bind to particular antigen, or other information.

In various embodiments, step 1008 includes selecting a representation indicating different subsets of individual immune cells within the clonotype group, wherein at least one (or each) different subset binds to a different combination of antigens. For example, step 1008 may include identifying a different interaction representation for at least one (or each) interaction associated with a subset of cells binding to a particular combination of antigens.

Step 1010 may include rendering a visualization of the clonotype group according to the visualization schema. In step 1010, the visualization visually presents or displays the at least one interaction. In various embodiments, step 1010 may be performed using at least one of visualization engine 319 or display unit 306 in FIG. 3.

In one or more other embodiments, method 1000 may include other steps such as, for example, without limitation, obtaining additional clonotype groups, wherein an additional clonotype group (or each clonotype group) comprises a set of individual immune cells; and rendering an additional visualization for the (or each) additional clonotype group according to the visualization schema.

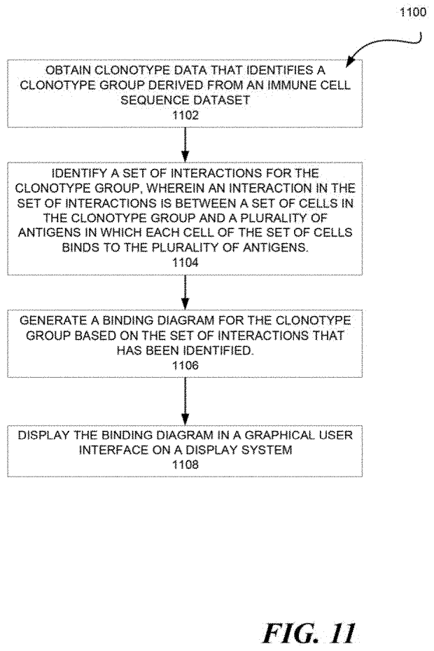

FIG. 11 is a flowchart of a method 1100 for visually presenting antigen binding profiles of cells in accordance with one or more embodiments. Method 1100 may be implemented using, for example, without limitation, visualization system 300 in FIG. 3.

Step 1102 includes obtaining clonotype data that identifies a clonotype group derived from an immune cell sequence dataset. The clonotype data may be, for example, clonotype data 310 in FIG. 3. The clonotype group may be, for example, clonotype group 321 in FIG. 3. In various embodiments, the clonotype data includes antigen binding information for the various cells belonging to the clonotype group.

Step 1104 includes identifying a set of interactions for the clonotype group, wherein an interaction in the set of interactions is between a set of cells in the clonotype group and a plurality of antigens in which a cell (or each cell) of the set of cells binds to the plurality of antigens. The set of interactions may be, for example, set of interactions 323 in FIG. 3. In one or more embodiments, the cells in the clonotype group are T cells. In other embodiments, the cells may be some other type of immune cells.

Step 1106 includes generating a binding diagram for the clonotype group based on the set of interactions that has been identified. Binding diagram 400 shown in FIG. 4-6 is one example of an implementation for the binding diagram created in step 1106. In some embodiments, step 1106 includes selecting the visualization schema for generating the binding diagram and then creating the binding diagram based on the visualization schema. In other embodiments, step 1104 described above includes identifying a visualization schema for representing the set of interactions and step 1106 includes generating the binding diagram based on this visualization schema.

The visualization schema, and thereby the binding diagram, may include at least a set of interaction representations that visually represents the set of interactions for the clonotype group. The set of interaction representations may take the form of, for example, set of interaction representations 326 in FIG. 3. Further, set of interaction representations 600 in FIG. 6 may be one example of an implementation for the set of interaction representations included in the visualization schema. An interaction representation in the set of interaction representations visually relates the plurality of antigens and visually indicates a number of cells in the set of cells that bind to the plurality of antigens. Visually relating the plurality of antigens may include, for example, graphically connecting (e.g., via edges, lines, curves, etc.) the plurality of antigens to show that the corresponding subset of immune cells binds to an antigen (or each antigen) of the plurality of antigens.

In addition to the set of interaction representations, the visualization schema may also include a spatial arrangement for presenting the antigens of interest and a set of graphical features corresponding to at least a portion of the antigens of interest. Spatial arrangement 406 in FIGS. 4-6 may be one example of an implementation for a spatial arrangement in the visualization schema. Set of graphical features 500 in FIGS. 5-6 may be one example of an implementation for the set of graphical features included in the visualization schema.

Step 1108 includes displaying the binding diagram in a graphical user interface on a display system. The graphical user interface may, in some cases, enable a user to interact with the binding diagram to identify further information about the multi-antigen binding of the cells in the clonotype group.

### VI. Computer System

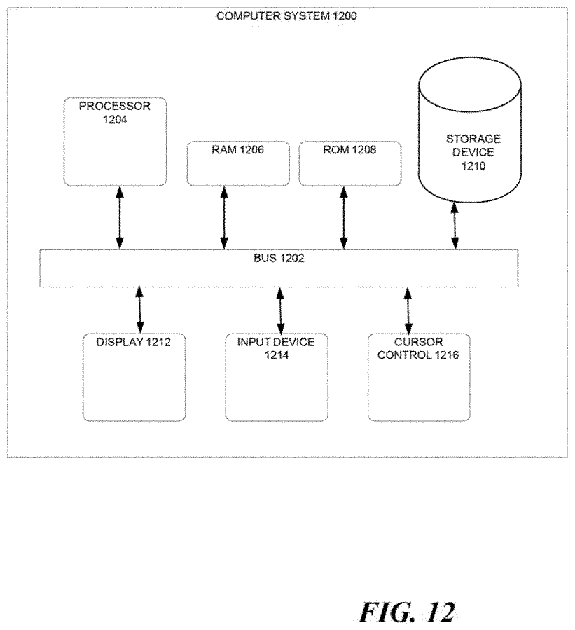

FIG. 12 is a block diagram that illustrates a computer system 1200 in accordance with various embodiments. In one or more embodiments, computer system 1200 may be used to implement computing platform 142 in FIG. 1, computing platform 302 in FIG. 3, or both.

In various embodiments, computer system 1200 can include a bus 1202 or other communication mechanism for communicating information, and a processor 1204 coupled with bus 1202 for processing information. In various embodiments, computer system 1200 can also include a memory, which can be a random-access memory (RAM) 1206 or other dynamic storage device, coupled to bus 1202 for determining instructions to be executed by processor 1204. Memory also can be used for storing temporary variables or other intermediate information during execution of instructions to be executed by processor 1204. In various embodiments, computer system 1200 can further include a read only memory (ROM) 1208 or other static storage device coupled to bus 1202 for storing static information and instructions for processor 1204. A storage device 1210, such as a magnetic disk or optical disk, can be provided and coupled to bus 1202 for storing information and instructions.

In various embodiments, computer system 1200 can be coupled via bus 1202 to a display 1212, such as a cathode ray tube (CRT) or liquid crystal display (LCD), for displaying information to a computer user. An input device 1214, including alphanumeric and other keys, can be coupled to bus 1202 for communicating information and command selections to processor 1204. Another type of user input device is a cursor control 1216, such as a mouse, a trackball or cursor direction keys for communicating direction information and command selections to processor 1204 and for controlling cursor movement on display 1212. This input device 1214 typically has two degrees of freedom in two axes, a first axis (i.e., x) and a second axis (i.e., y), that allows the device to specify positions in a plane. However, it should be understood that input devices 1214 allowing for 3-dimensional (x, y and z) cursor movement are also contemplated herein.

Consistent with certain implementations of the embodiments described herein, results can be provided by computer system 1200 in response to processor 1204 executing one or more sequences of one or more instructions contained in memory 1206. Such instructions can be read into memory 1206 from another computer-readable medium or computer-readable storage medium, such as storage device 1210. Execution of the sequences of instructions contained in memory 1206 can cause processor 1204 to perform the processes described herein. Alternatively, hard-wired circuitry can be used in place of or in combination with software instructions to implement embodiments described herein. Thus, implementations of the embodiments described herein are not limited to any specific combination of hardware circuitry and software.

The term “computer-readable medium” (e.g., data store, data storage, etc.) or “computer-readable storage medium” as used herein refers to any media that participates in providing instructions to processor 1204 for execution. Such a medium can take many forms, including but not limited to, non-volatile media, volatile media, and transmission media. Examples of non-volatile media can include, but are not limited to, optical, solid state, magnetic disks, such as storage device 1210. Examples of volatile media can include, but are not limited to, dynamic memory, such as memory 1206. Examples of transmission media can include, but are not limited to, coaxial cables, copper wire, and fiber optics, including the wires that comprise bus 1202.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1204 of computer system 1200 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 1200 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods described herein may be implemented as firmware and/or a software program and applications written in conventional programming languages such as C, C++, Python, Rust, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1200 of Appendix D, whereby processor 1204 would execute the analyses and determinations provided by these engines, subject to instructions provided by any one of, or a combination of, memory components 1206/408/410 and user input provided via input device 1214.

Digital Processing Device

In various embodiments, the systems and methods described herein can include a digital processing device or use of the same. In various embodiments, the digital processing device can include one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In some embodiments, the non-volatile memory comprises dynamic random-access memory (DRAM). In various embodiments, the non-volatile memory comprises ferroelectric random-access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing-based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

Computer Program

In various embodiments, the systems and methods disclosed herein can include at least one computer program or use at least one computer program. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® .NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or eXtensible Markup Language (XML). In various embodiments, a web application is written to some extent in a presentation definition language such as Cascading Style Sheets (CSS). In various embodiments, a web application is written to some extent in a client-side scripting language such as Asynchronous Javascript and XML (AJAX), Flash® Actionscript, Javascript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™ and Unity®.

Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Frame-work, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo DSi Shop.

Standalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Rust, Objective-C, COBOL, Delphi, Eiffel, Java™, Lisp, Python™, Visual Basic, and VB.NET, or combinations thereof. Compilation is often performed, at least in part, to create an executable program. In various embodiments, a computer program includes one or more executable complied applications.

Web Browser Plug-In

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, Rust, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire-fox®, Google® Chrome, Apple® Safari®, Opera Software® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object-oriented databases, object databases, entity-relation-ship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

### VII. Recitation of Embodiments

Embodiment 1. A method for visualizing immune cells within an immune cell receptor dataset, the method comprising:

obtaining the immune cell receptor dataset from a sample, the immune cell receptor dataset including a plurality of immune cell receptor sequences, wherein each immune cell receptor sequence is associated with an individual immune cell in the sample;

obtaining a clonotype group comprising a set of individual immune cells from the immune cell receptor dataset;

identifying at least one interaction between at least one cell of the set of individual immune cells in the clonotype group and a plurality of antigens;

selecting a visualization schema to visualize the at least one interaction; and

rendering a visualization of the clonotype group according to the visualization schema, wherein the visualization displays the at least one interaction.

Embodiment 2. The method of Embodiment 1, wherein the at least one interaction comprises a plurality of interactions between at least one cell of the set of individual immune cells in the clonotype group and the plurality of antigens.

Embodiment 3. The method of Embodiment 2, wherein the at least one interaction comprises a plurality of interactions between a plurality of cells of the set of individual immune cells in the clonotype group and the plurality of antigens.

Embodiment 4. The method of Embodiment 1, wherein the sample includes cells taken from at least one of a diseased subject, a convalescent subject, a vaccinated subject, a healthy subject, an immunosuppressed subject, or a subject having an autoimmune disorder.

Embodiment 5. The method of Embodiment 1, wherein each immune cell receptor sequence comprises at least one heavy chain region sequence and at least one light chain region sequence.

Embodiment 6. The method of Embodiment 1, wherein obtaining the clonotype group comprises:

comparing the immune cell receptor sequences associated with a first immune cell and a second immune cell from the sample; and

identifying the first immune cell and the second immune cell as members of a same clonotype if one or more immune cell receptor sequence comparison criteria is met.

Embodiment 7. The method of Embodiment 1, wherein the immune cell receptor dataset comprises antigen binding information for the plurality of immune cell receptor sequences, and wherein identifying the at least one interaction between the at least one cell of the set of individual immune cells in the clonotype group and the plurality of antigens comprises using the antigen binding information to identify the at least one interaction.

Embodiment 8. The method of Embodiment 7, wherein identifying the at least one interaction between the at least one cell of the set of individual immune cells in the clonotype group and the plurality of antigens comprises determining the at least one cell as binding to an antigen if the at least one cell has at least a pre-determined number of copies of immune cell receptor sequences that bind the antigen.

Embodiment 9. The method of Embodiment 1, wherein selecting the visualization schema comprises:

selecting a spatial arrangement of the plurality of antigens based on a relationship between first human leukocyte antigen (HLA) alleles present in the plurality of antigens and second HLA alleles expressed by the immune cells of the sample.

Embodiment 10. The method of Embodiment 1, wherein selecting the visualization schema comprises:

selecting a representation for each of the plurality of antigens, wherein the representation for a particular antigen of the plurality of antigens represents information of at least one of a number of cells in the clonotype group that bind to the particular antigen or a proportion of cells in the clonotype group that bind to the particular antigen.

Embodiment 11. The method of Embodiment 1, wherein selecting the visualization schema comprises:

selecting a representation indicating different subsets of individual immune cells within the clonotype group, wherein each different subset binds to a different combination of antigens.

Embodiment 12. The method of Embodiment 1, further comprising obtaining additional clonotype groups, each additional clonotype group comprising a distinct set of individual immune cells; and

rendering an additional visualization for each additional clonotype group according to the visualization schema.

Embodiment 13. A computer-program product tangibly embodied in a non-transitory machine-readable storage medium, including instructions configured to cause one or more data processors to perform a method for visualizing immune cells within an immune cell receptor dataset, the method comprising:

obtaining the immune cell receptor dataset from a sample, the immune cell receptor dataset including a plurality of immune cell receptor sequences, wherein each immune cell receptor sequence is associated with an individual immune cell in the sample;

obtaining a clonotype group comprising a set of individual immune cells from the immune cell receptor dataset;

identifying at least one interaction between at least one cell of the set of individual immune cells in the clonotype group and a plurality of antigens;

selecting a visualization schema to visualize the at least one interaction; and

rendering a visualization of the clonotype group according to the visualization schema, wherein the visualization displays the at least one interaction.

Embodiment 14. The computer-program product of Embodiment 13, wherein the at least one interaction comprises a plurality of interactions between at least one cell of the set of individual immune cells in the clonotype group and the plurality of antigens.

Embodiment 15. The computer-program product of Embodiment 14, wherein the at least one interaction comprises a plurality of interactions between a plurality of cells of the set of individual immune cells in the clonotype group and the plurality of antigens.

Embodiment 16. The computer-program product of Embodiment 13, wherein the sample includes cells taken from at least one of a diseased subject, a convalescent subject, a vaccinated subject, a healthy subject, an immunosuppressed subject, or a subject having an autoimmune disorder.

Embodiment 17. The computer-program product of Embodiment 13, wherein each immune cell receptor sequence comprises at least one heavy chain region sequence and at least one light chain region sequence.

Embodiment 18. The computer-program product of Embodiment 13, wherein the method further comprises:

comparing the immune cell receptor sequences associated with a first immune cell and a second immune cell from the sample; and

identifying the first immune cell and the second immune cell as members of a same clonotype if one or more immune cell receptor sequence comparison criteria is met.

Embodiment 19. The computer-program product of Embodiment 13, wherein the immune cell receptor dataset comprises antigen binding information for the plurality of immune cell receptor sequences, and wherein identifying the at least one interaction between the at least one cell of the set of individual immune cells in the clonotype group and the plurality of antigens comprises using the antigen binding information to identify the at least one interaction.

Embodiment 20. The computer-program product of Embodiment 19, wherein identifying the at least one interaction between the at least one cell of the set of individual immune cells in the clonotype group and the plurality of antigens comprises determining the at least one cell as binding to an antigen if the at least one cell has at least a pre-determined number of copies of immune cell receptor sequences that bind the antigen.

Embodiment 21. The computer-program product of Embodiment 13, wherein the method further comprises:

selecting a spatial arrangement of the plurality of antigens based on a relationship between first human leukocyte antigen (HLA) alleles present in the plurality of antigens and second HLA alleles expressed by the immune cells of the sample.

Embodiment 22. The computer-program product of Embodiment 13, wherein the method further comprises:

selecting a representation for each of the plurality of antigens, wherein the representation for a particular antigen of the plurality of antigens represents information of at least one of a number of cells in the clonotype group that bind to the particular antigen or a proportion of cells in the clonotype group that bind to the particular antigen.

Embodiment 23. The computer-program product of Embodiment 13, wherein the method further comprises:

selecting a representation indicating different subsets of individual immune cells within the clonotype group, wherein each different subset binds to a different combination of antigens.

Embodiment 24. The computer-program product of Embodiment 13, wherein the method further comprises:

obtaining additional clonotype groups, each additional clonotype group comprising a distinct set of individual immune cells; and

rendering an additional visualization for each additional clonotype group according to the visualization schema.

Embodiment 25. A visualization system comprising:

a data source configured to obtain an immune cell receptor dataset from a sample, the immune cell receptor dataset including a plurality of immune cell receptor sequences, wherein each immune cell receptor sequence is associated with an individual immune cell in the sample;

a processing unit communicatively connected to the data source and configured to receive the immune cell receptor dataset, the processing unit comprising:
a clonotype grouping engine configured to obtain a clonotype group comprising a set of individual immune cells from the immune cell receptor dataset;
an interaction identification engine configured to identify at least one interaction between at least one cell of the set of individual immune cells in the clonotype group and a plurality of antigens; and
a schema selection engine configured to select a visualization schema to visualize the at least one interaction; and

a display unit configured to render a visualization of the clonotype group according to the visualization schema, wherein the visualization displays the at least one interaction.

Embodiment 26. The visualization system of Embodiment 25, wherein the at least one interaction comprises a plurality of interactions between at least one cell of the set of individual immune cells in the clonotype group and the plurality of antigens.

Embodiment 27. The visualization system of Embodiment 26, wherein the at least one interaction comprises a plurality of interactions between a plurality of cells of the set of individual immune cells in the clonotype group and the plurality of antigens.

Embodiment 28. The visualization system of Embodiment 25, wherein the sample includes cells taken from at least one of a diseased subject, a convalescent subject, a vaccinated subject, a healthy subject, an immunosuppressed subject, or a subject having an autoimmune disorder.

Embodiment 29. The visualization system of Embodiment 25, wherein each immune cell receptor sequence comprises at least one heavy chain region sequence and at least one light chain region sequence.

Embodiment 30. The visualization system of Embodiment 25, wherein the clonotype grouping engine is configured to compare the immune cell receptor sequences associated with a first immune cell and a second immune cell from the sample; and identify the first immune cell and the second immune cell as members of a same clonotype if one or more immune cell receptor sequence comparison criteria is met.

Embodiment 31. The visualization system of Embodiment 25, wherein the immune cell receptor dataset comprises antigen binding information for the plurality of immune cell receptor sequences, and wherein the interaction identification engine is configured to use the antigen binding information to identify the at least one interaction.

Embodiment 32. The visualization system of Embodiment 31, wherein the interaction identification engine is configured to determine the at least one cell as binding to an antigen if the at least one cell has at least a pre-determined number of copies of immune cell receptor sequences that bind the antigen.

Embodiment 33. The visualization system of Embodiment 25, wherein schema selection engine is configured to select a spatial arrangement of the plurality of antigens based on a relationship between first human leukocyte antigen (HLA) alleles present in the plurality of antigens and second HLA alleles expressed by the immune cells of the sample.

Embodiment 34. The visualization system of Embodiment 25, wherein schema selection engine is configured to select a representation for each of the plurality of antigens, wherein the representation for a particular antigen of the plurality of antigens represents information of at least one of a number of cells in the clonotype group that bind to the particular antigen or a proportion of cells in the clonotype group that bind to the particular antigen.

Embodiment 35. The visualization system of Embodiment 25, wherein schema selection engine is configured to select a representation indicating different subsets of individual immune cells within the clonotype group, wherein each different subset binds to a different combination of antigens.

Embodiment 36. The visualization system of Embodiment 25, wherein the clonotype grouping engine is configured to obtain additional clonotype groups, each additional clonotype group comprising a distinct set of individual immune cells; and wherein the display unit is configured render an additional visualization for each additional clonotype group according to the visualization schema.

Embodiment 37. A method for visualizing multi-antigen binding capabilities of a set of clonotype groups, the method comprising:

obtaining clonotype data that identifies a clonotype group derived from an immune cell sequence dataset;

identifying a set of interactions for the clonotype group, wherein an interaction in the set of interactions is between a set of cells in the clonotype group and a plurality of antigens in which each cell of the set of cells binds to the plurality of antigens;

generating a binding diagram for the clonotype group based on the set of interactions that has been identified,
wherein the binding diagram includes a set of interaction representations that visually represents the set of interactions for the clonotype group; and
wherein an interaction representation in the set of interaction representations visually relates the plurality of antigens and visually indicates a number of cells in the set of cells that bind to the plurality of antigens.

Embodiment 38. The method of Embodiment 37, further comprising:

displaying the binding diagram in a graphical user interface on a display system.

Embodiment 39. The method of Embodiment 37, wherein the binding diagram includes a graphical feature for a corresponding antigen that provides a visual indication of at least one of a number of cells in the clonotype group that bind to the corresponding antigen or a proportion of cells in the clonotype group that bind to the corresponding antigen.

Embodiment 40. The method of Embodiment 37, wherein the graphical feature is a shape indicator having a size that visually indicates the number of cells in the clonotype group that bind to the corresponding antigen and at least one of a color, a shade, a texture, or a pattern that visually indicates the proportion of cells in the clonotype group that bind to the corresponding antigen.

Embodiment 41. The method of Embodiment 37, wherein generating the binding diagram comprises:

selecting a visualization schema that includes the set of interaction representations.

Embodiment 42. The method of Embodiment 37, wherein the interaction representation includes a set of edges with each edge of the set of edges connecting two antigens of the plurality of antigens.

Embodiment 43. The method of Embodiment 37, wherein the interaction representation includes a set of curves and wherein each curve in the set of curves has a same thickness that indicates the number of cells in the set of cells that bind to the plurality of antigens.

Embodiment 44. The method of Embodiment 37, wherein the binding diagram includes a spatial arrangement of a collection of antigens that are of interest based on a matching of first human leukocyte antigen (HLA) alleles present in the collection of antigens to second HLA alleles expressed by immune cells in the clonotype group.

Embodiment 45. The method of Embodiment 37, further comprising: displaying the binding diagram in a graphical user interface on a display system along with at least one other binding diagram that corresponds to a different clonotype group.

Embodiment 46. A computer-program product tangibly embodied in a non-transitory machine-readable storage medium, including instructions configured to cause one or more data processors to perform the method of one or more of embodiments 1-12 and 37-45.

Embodiment 47. A visualization system comprising:

a data source configured to obtain an immune cell receptor dataset from a sample, the immune cell receptor dataset including a plurality of immune cell receptor sequences, wherein each immune cell receptor sequence is associated with an individual immune cell in the sample; and

a processing unit communicatively connected to the data source and configured to receive the immune cell receptor dataset, the processing unit configured to perform the method of one or more of embodiments 37-45.

Embodiment 48. A method comprising the method of Embodiment 37 and one or more embodiments 38-45.

Embodiment 49. The method of any one of Embodiments 1-12, wherein the sample comprises a tissue sample and the immune cell receptor dataset comprises a spatial dataset obtained from the tissue sample.

Embodiment 50. The computer-program product of any one of Embodiments 13-24, wherein the sample comprises a tissue sample and the immune cell receptor dataset comprises a spatial dataset obtained from the tissue sample.

Embodiment 51. The system of any one of Embodiments 25-36, wherein the sample comprises a tissue sample and the immune cell receptor dataset comprises a spatial dataset obtained from the tissue sample.

Embodiment 52. The method of any one of Embodiments 37-45, wherein the immune cell receptor dataset comprises a spatial dataset obtained from a sample, wherein the sample comprises a tissue sample.

Embodiment 53. The computer-program product of Embodiment 46, wherein the sample comprises a tissue sample and the immune cell receptor dataset comprises a spatial dataset obtained from the tissue sample

Embodiment 54. The system of Embodiment 47, wherein the sample comprises a tissue sample and the immune cell receptor dataset comprises a spatial dataset obtained from the tissue sample.

Embodiment 55. The method of Embodiment 48, wherein the immune cell receptor dataset comprises a spatial dataset obtained from a sample, wherein the sample comprises a tissue sample.

1. A method for visualizing immune cells within an immune cell receptor dataset, the method comprising:
obtaining the immune cell receptor dataset from a sample, the immune cell receptor dataset including a plurality of immune cell receptor sequences, wherein each immune cell receptor sequence is associated with an individual immune cell in the sample;
obtaining a clonotype group comprising a set of individual immune cells from the immune cell receptor dataset;
identifying at least one interaction between at least one cell of the set of individual immune cells in the clonotype group and a plurality of antigens;
selecting a visualization schema to visualize the at least one interaction; and
rendering a visualization of the clonotype group according to the visualization schema, wherein the visualization displays the at least one interaction.

2. The method of claim 1, wherein the at least one interaction comprises a plurality of interactions between at least one cell of the set of individual immune cells in the clonotype group and the plurality of antigens.

3. The method of claim 2, wherein the at least one interaction comprises a plurality of interactions between a plurality of cells of the set of individual immune cells in the clonotype group and the plurality of antigens.

4. (canceled)

5. The method of claim 1, wherein each immune cell receptor sequence comprises at least one heavy chain region sequence and at least one light chain region sequence.

6. The method of claim 1, wherein obtaining the clonotype group comprises:
comparing the immune cell receptor sequences associated with a first immune cell and a second immune cell from the sample; and
identifying the first immune cell and the second immune cell as members of a same clonotype if one or more immune cell receptor sequence comparison criteria is met.

7. The method of claim 1, wherein the immune cell receptor dataset comprises antigen binding information for the plurality of immune cell receptor sequences, and wherein identifying the at least one interaction between the at least one cell of the set of individual immune cells in the clonotype group and the plurality of antigens comprises using the antigen binding information to identify the at least one interaction.

8. The method of claim 7, wherein identifying the at least one interaction between the at least one cell of the set of individual immune cells in the clonotype group and the plurality of antigens comprises determining the at least one cell as binding to an antigen if the at least one cell has at least a pre-determined number of copies of immune cell receptor sequences that bind the antigen.

9. The method of claim 1, wherein selecting the visualization schema comprises:
selecting a spatial arrangement of the plurality of antigens based on a relationship between first human leukocyte antigen (HLA) alleles present in the plurality of antigens and second HLA alleles expressed by the immune cells of the sample.

10. The method of claim 1, wherein selecting the visualization schema comprises:
selecting a representation for each of the plurality of antigens, wherein the representation for a particular antigen of the plurality of antigens represents information of at least one of a number of cells in the clonotype group that bind to the particular antigen or a proportion of cells in the clonotype group that bind to the particular antigen.

11. The method of claim 1, wherein selecting the visualization schema comprises:
selecting a representation indicating different subsets of individual immune cells within the clonotype group, wherein each different subset binds to a different combination of antigens.

12. The method of claim 1, further comprising
obtaining additional clonotype groups, each additional clonotype group comprising a distinct set of individual immune cells; and
rendering an additional visualization for each additional clonotype group according to the visualization schema.

13.-36. (canceled)

37. A method for visualizing multi-antigen binding capabilities of a set of clonotype groups, the method comprising:
obtaining clonotype data that identifies a clonotype group derived from an immune cell sequence dataset;
identifying a set of interactions for the clonotype group, wherein an interaction in the set of interactions is between a set of cells in the clonotype group and a plurality of antigens in which each cell of the set of cells binds to the plurality of antigens;
generating a binding diagram for the clonotype group based on the set of interactions that has been identified,
wherein the binding diagram includes a set of interaction representations that visually represents the set of interactions for the clonotype group; and
wherein an interaction representation in the set of interaction representations visually relates the plurality of antigens and visually indicates a number of cells in the set of cells that bind to the plurality of antigens.

38. The method of claim 37, further comprising:
displaying the binding diagram in a graphical user interface on a display system.

39. The method of claim 37, wherein the binding diagram includes a graphical feature for a corresponding antigen that provides a visual indication of at least one of a number of cells in the clonotype group that bind to the corresponding antigen or a proportion of cells in the clonotype group that bind to the corresponding antigen.

40. The method of claim 37, wherein the graphical feature is a shape indicator having a size that visually indicates the number of cells in the clonotype group that bind to the corresponding antigen and at least one of a color, a shade, a texture, or a pattern that visually indicates the proportion of cells in the clonotype group that bind to the corresponding antigen.

41. The method of claim 37, wherein generating the binding diagram comprises:
selecting a visualization schema that includes the set of interaction representations.

42. The method of claim 37, wherein the interaction representation includes a set of edges with each edge of the set of edges connecting two antigens of the plurality of antigens.

43. The method of claim 37, wherein the interaction representation includes a set of curves and wherein each curve in the set of curves has a same thickness that indicates the number of cells in the set of cells that bind to the plurality of antigens.

44. The method of claim 37, wherein the binding diagram includes a spatial arrangement of a collection of antigens that are of interest based on a matching of first human leukocyte antigen (HLA) alleles present in the collection of antigens to second HLA alleles expressed by immune cells in the clonotype group.

45. The method of claim 37, further comprising:
displaying the binding diagram in a graphical user interface on a display system along with at least one other binding diagram that corresponds to a different clonotype group.
